(19) United States
(12) Patent Application Publication
(10) Pub. No.: US 2012/0142731 A1
(43) Pub. Date: Jun. 7, 2012

(54) METHODS OF INHIBITING VIRAL INFECTION

(75) Inventors: Michael Kinch, Laytonsville, MD (US); Michael Goldblatt, McLean, VA (US)

(73) Assignee: Functional Genetics, Inc., Gaithersburg, MD (US)

(21) Appl. No.: 12/261,603

(22) Filed: Oct. 30, 2008

Related U.S. Application Data
(60) Provisional application No. 60/983,966, filed on Oct. 31, 2007.

(51) Int. Cl. A61K 31/4706; A61P 31/12; C12N 5/071; A61P 31/14; A61P 31/16; C07D 215/46; A61P 31/18

(52) U.S. Cl. 514/313; 546/160; 435/375

(57) ABSTRACT

Compounds, pharmaceutical compositions and methods of inhibiting viral infection in a mammal in need of same, are provided, which employ compounds of the formula

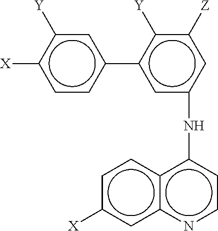

wherein each X is independently H or an electrodonating group, each Y is independently H, alkyl of 1-4 carbon atoms, hydroxy, alkoxy or methylene and wherein Substituent Z is a di-or-tri akly amino, or alkyl di or tri amino, optionally substituted with a halogen moiety. This family of compounds, designated FGI-104 herein, inhibits viral infection therapeutically and prophylactically.

### PRIORITY DATA AND INCORPORATION BY REFERENCE

This application claims benefit of priority to U.S. Provisional Patent Application No. 60/983,966 filed Oct. 31, 2007, which is incorporated herein by reference in its entirety.

### BACKGROUND OF THE INVENTION

### Background of the Prior Art

Humans, and mammals in general, including commercially important mammals such as pigs, cows and sheep, as well as higher mammals such as monkeys, are subject to infection by a wide variety of viruses. These viruses vary markedly in structure, life cycle, susceptible cells and animal targets, and the like. Many times, a vaccination or treatment method to limit or inhibit viral infection is time limited—a first generation of virus may be effectively inhibited, only to have a mutated strain break any protection conferred by an earlier vaccine or therapy.

It would be desirable to identify agents that inhibit more than one specific viral agent, so that different viral infections could be inhibited with a single agent or family of agents, and not be defeated by the frequent mutations exhibited by the viral population. Until now, given the extreme variety of viral infectious modes and characteristics across the various viral families, it has been difficult to establish a common therapy. One pathway, apparently mediated by TSG101, is the subject of ongoing studies. Inhibition via this pathway, however, requires the generation of selective antibodies, and no specific agent or composition has been identified to interfere with these pathways on a commercial basis. These efforts are discussed, inter alia, in U.S. Pat. Nos. 6,835,816 and 6,248,523.

U.S. patent application Ser. No. 11/166,726, filed Jun. 27, 2005 and published as 2006-0142259 A1, the entirety of which is incorporated by reference includes a set of compounds that were found to have activity in blocking TSG101 binding to the PTAP domain found in the HIV p6 Gag protein. Given the role of TSG101 in HIV infectivity, these compounds were indicated to have potential therapeutic utility for HIV/AIDS, as reflected by in vitro testing.

In the period 1979-1981, the Walter Reed Army Institute of Research sponsored studies conducted in the main part by Hazleton Laboratories America, Inc. then of Vienna, Va., to evaluate the efficacy and safety of the use of a compound designated WR-228,258 2HCl as an anti-malarial agent for use in the military. The principal contract number was DAMD-17-80-C-0161. These studies were never released to the public, and remain confidential, and proprietary to the assignee of this application, except to the extent described herein. The compound was shown to be well tolerated and suitable for oral and IP, as well as IV administration, and effective against the parasite based disease at about the same level as the dominant treatment, chloroquinoline. It was not effective against chloroquinoline-resistant malarial strains, however, and was ultimately abandoned. The compound tested had the chemical name, as set forth in the reports, 4′-chloro-5-[(7-chloro-4-quinolinyl)amino]-3-(1,1-dimethylethylamino)methyl[1,1′-biphenyl]-2-ol dihydrochloride. The compound was administered as a salt in dosage values of from 9-1250 mg (as a salt)/kg of body weight. Mammalian models used were dogs, rabbits, mice, and rats.

Given the developing knowledge of the fundamental role played by TSG101 and other ESCRT proteins as “hijacked” vehicles to carry viral particles to the infected cell surface to complete maturation, bud and release, see U.S. patent application Ser. No. 11/939,122, incorporated herein-by-reference, a group of compounds was designed to potentially inhibit or interfere with the interaction between TSG101 and viral particles. This family was subsequently tested for anti-viral activity, and the most promising candidates were identified, and subjected to further testing. One of the compounds so identified was 4′-chloro-5-[(7-chloro-4-quinolinyl)amino]-3-(1,1-dimethylethylamino)methyl [1,1′-biphenyl]-2-ol, the very compound studied 25 years previously for Walter Reed. This compound is referenced herein as R24.

### SUMMARY OF THE INVENTION

Additional testing addressing this panel of compounds, including lead compounds designated as R19 and R24, has shown this family of compounds to be effective in the inhibition of a broad spectrum of different viral pathogens. The compound family, as described below, is designated as FGI-104 compounds. These compounds were generated based on predicted inhibition of TSG101. While the activity of these molecules may not necessarily involve or be limited to TSG101 as a target, data generated does indicate that the FGI-104 compounds appear to inhibit viral activity by blocking late stage viral activity, possibly after completion of viral protein synthesis. This would be consistent with targeting TSG101, as interfering/inhibiting the interaction of the viral particles and this protein could interfere with travel by the virus to the cell surface and subsequent budding.

Compounds of particular interest include compounds R19, R24. Collectively, this family of compounds is referred to herein as FGI-104 compounds. The chemical formulae of compounds R19 and R24 are given in FIGS. 1-2.

### BRIEF DESCRIPTION OF THE DRAWINGS

FIG. 1 sets forth the Chemical Structure, Molecular Formula and IUPAC name, as well as pertinent physico-chemical properties of one of the active compounds that is the subject of this invention, R19.

FIG. 2 sets forth the Chemical Structure, Molecular Formula and IUPAC name, as well as pertinent physico-chemical properties one of the active compounds that is the subject of this invention, R24.

FIG. 3 presents the activity of R19 and R24 against infection challenge, in vitro, against HIV.

FIG. 4 presents the activity of R19 and R24 against infection challenge, in vitro, against Influenza.

FIG. 5 presents the activity of R19 and R24 against infection challenge, in vitro, against Hepatitis viruses (HBV, HCV 1, HCV 2).

FIG. 6 presents the activity of R19 and R24 against infection challenge, in vitro, against PRRS Virus, a viral infection most commonly found in pigs.

FIG. 7 provides a common chemical structure for compounds of this family showing antiviral activity

FIGS. 8A-8O depict various members of the family of small molecules of FGI-104.

FIGS. 9A and 9B present, in tabular form, the CC₅₀ and inhibitory dosage of the members of the FGI-104 family shown in FIGS. 8A-8P when measured against a single virus—porcine reproductive and respiratory syndrome (PRRS) virus, a commercially critical virus that devastates pig stocks worldwide.

FIG. 10 presents in capsule form one of the lead compounds of the FGI-104 chemical family, as well as its fundamental properties at a glance.

FIG. 11 presents, in tabular form, pertinent information as to the EC₅₀ and CC₅₀ for R24 when tested against a number of different viruses drawn from very different families.

FIG. 12 presents, in graph and table form, information regarding the inhibition of a pox virus by R24 in cell based assays.

FIG. 13 provides data in the form of a graph, table and protein blot confirming the inhibition of Hepatitis C virus by R24.

FIG. 14 provides by table and graph information reflecting the inhibition of Hepatitis B virus by R24.

FIG. 15 provides further information on PRRS virus inhibition by R24 in table form.

FIG. 16 provides a bar graph reflecting data showing the inhibition of influenza virus by R24.

FIG. 17 provides information by graph and table demonstrating the inhibition of Ebola hemorrhagic virus by R24.

FIG. 18 addresses inhibition of Venezuelan Equine Encephalomyelitis (VEE), an alpha virus, by R24.

The inhibition of HIV, perhaps the world's most targeted virus, is set forth by graph and table in FIG. 19.

FIG. 20 demonstrates, by comparing R24 inhibition of HBV in a viral release assay, and a replicon suppression assay, that R24 activity is present in the later stages of the viral cycle.

FIG. 21 presents in Table form information demonstrating R24 is not toxic to cells.

FIG. 22 presents in table and bullet point format certain information obtained from earlier studies on R24, demonstrating its safety for mammalian use.

FIG. 23 presents the chemical name, structure and physiochemical properties of lead compound R24.

FIG. 24 presents a simple synthesis scheme for lead compound R24 consistent with good laboratory practice.

FIG. 25 presents data on the efficiency and scalability of the synthesis scheme of FIG. 24.

FIG. 26 presents in table form the solubility of R24 in various pharmaceutically oriented solvent systems.

### DETAILED DESCRIPTION OF THE INVENTION

The family of FGI-104 compounds was developed by computer design to identify compounds that would interfere with late stage (in the virus maturation cycle) binding between TSG101 and target viruses. As only one example, TSG is known to bind with the PTAP motif of HIV. Compounds designed to fit and occupy the TSG101 binding site would have a reasonable expectation of preventing the binding between TSG101 and target viruses. As noted above, for any viruses, transport by TSG101 and related proteins of the EXCRT or ESCRT-1 complex have been shown to be a prerequisite to migration to the cell surface, maturation and budding. Without the TSG101 binding event, the expectation was that viral reproduction and spread of infection would be inhibited.

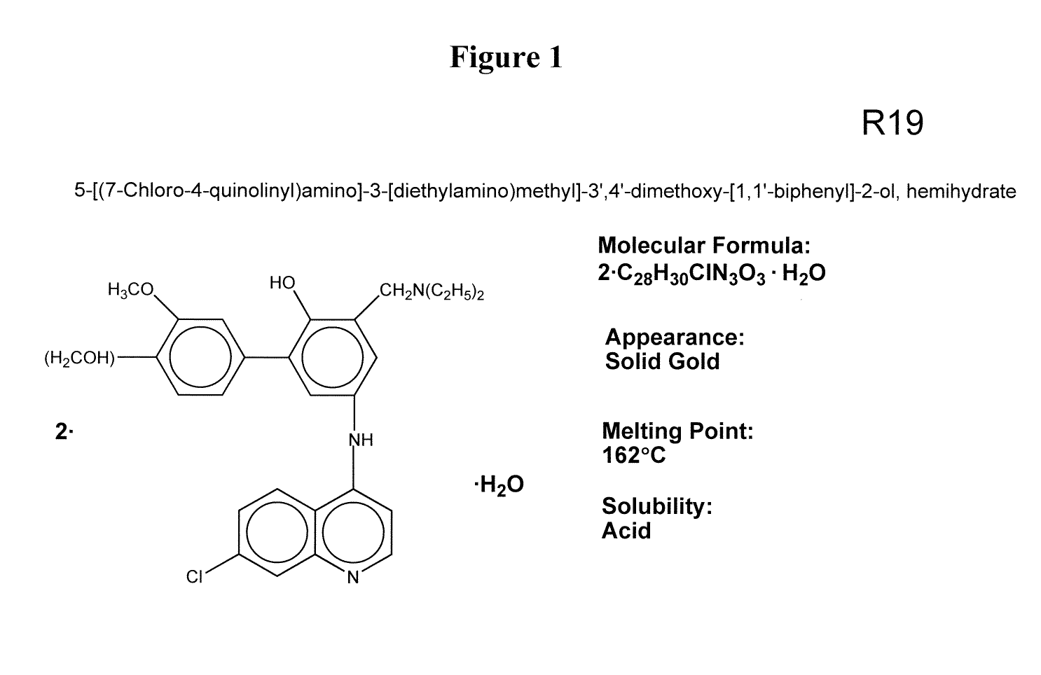

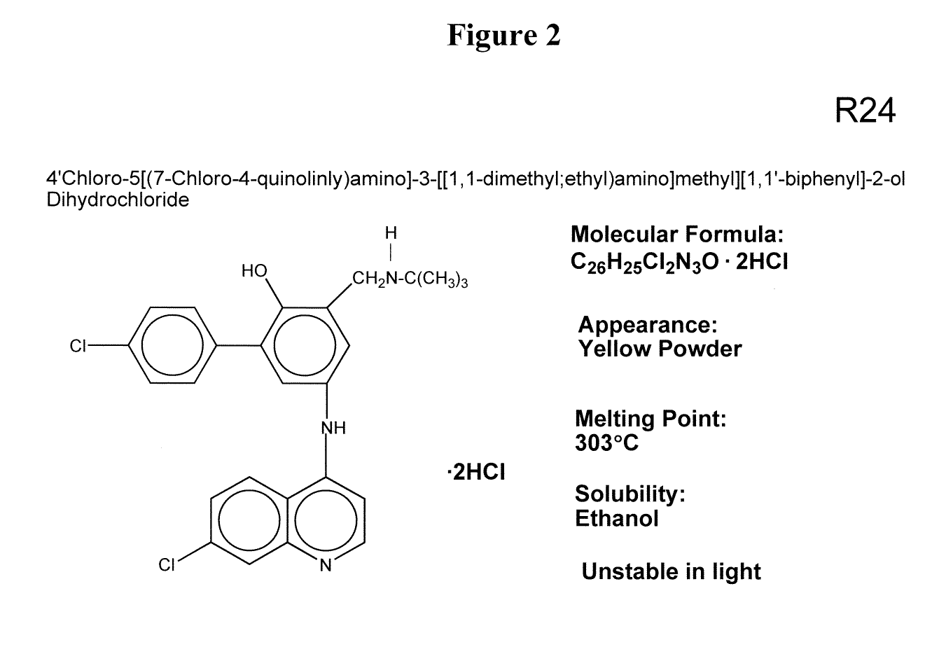

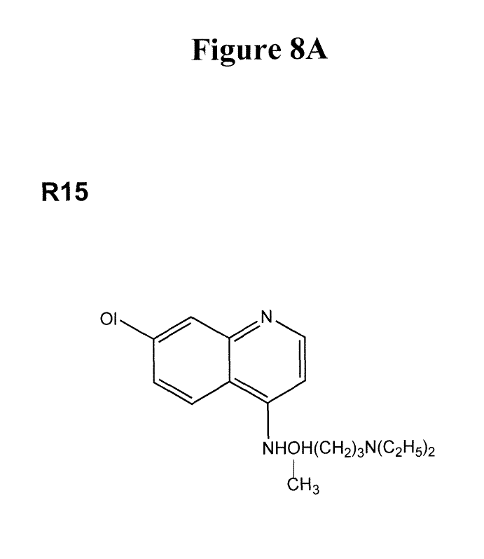

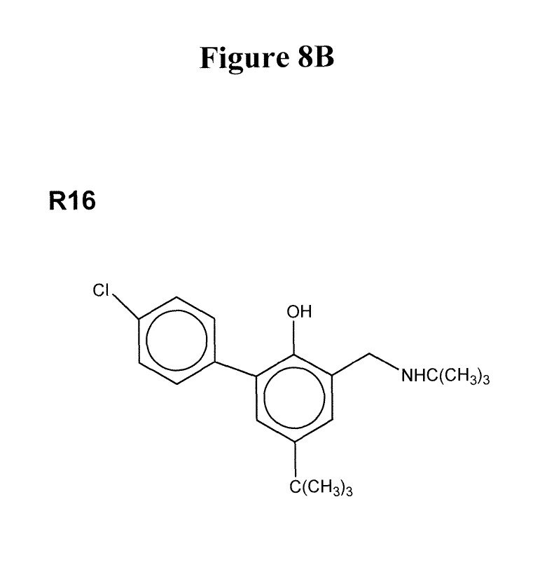

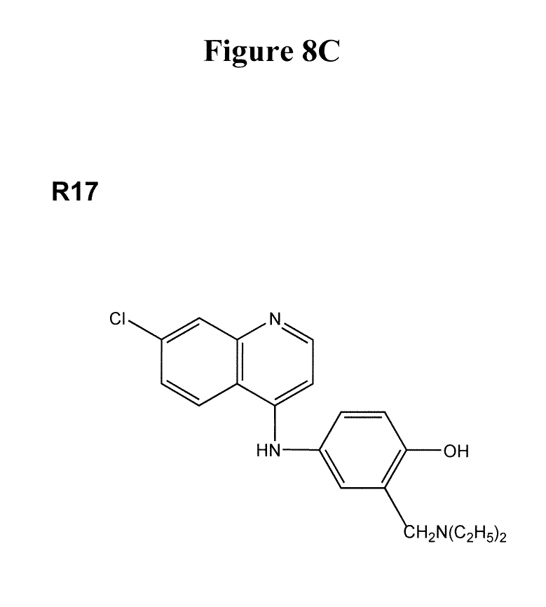

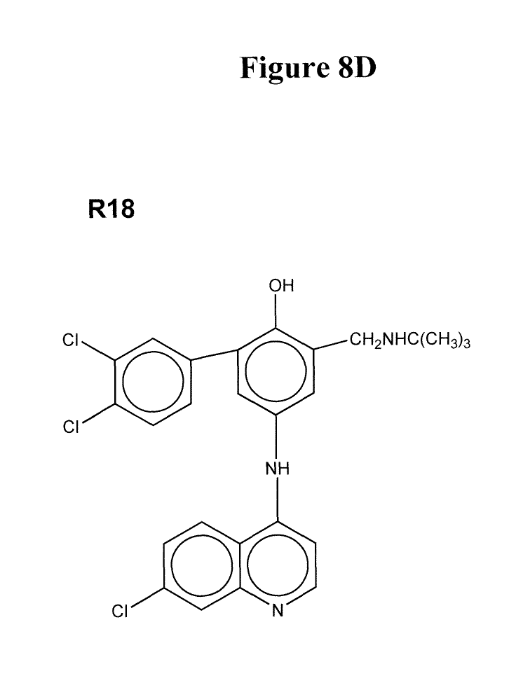

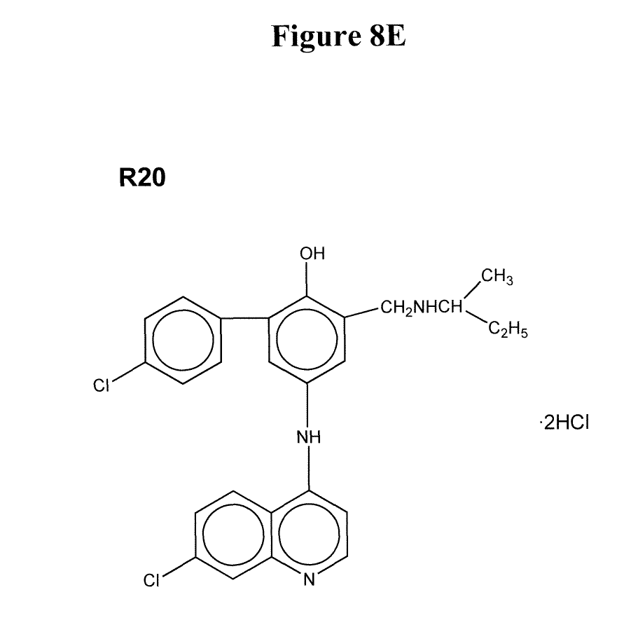

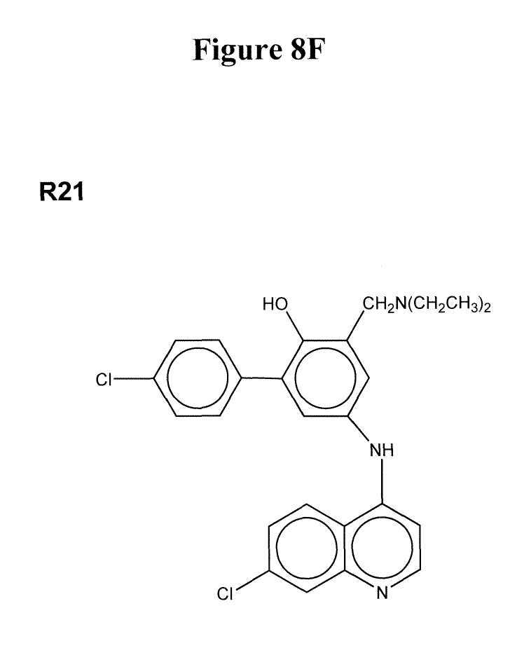

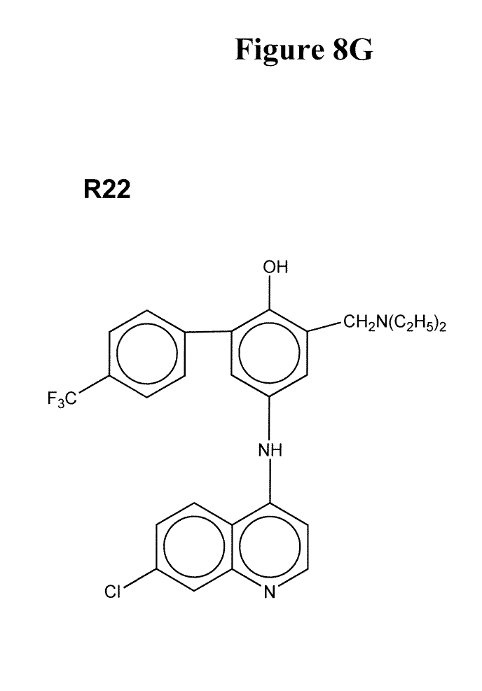

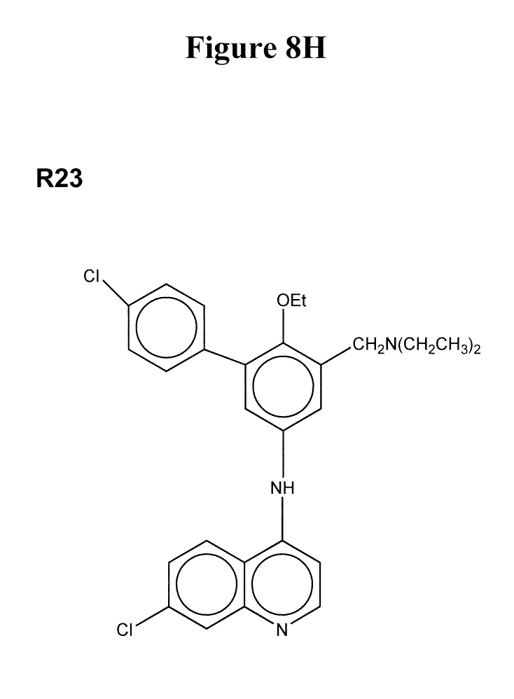

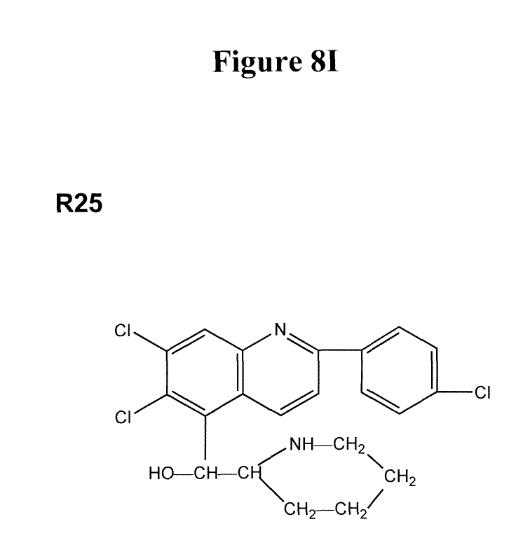

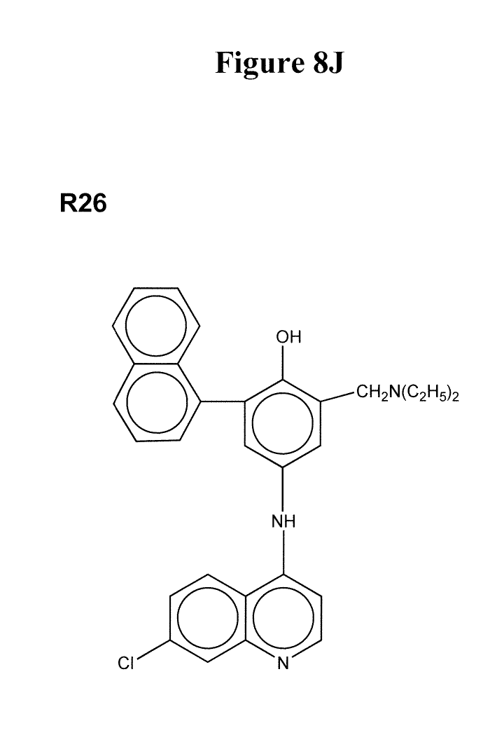

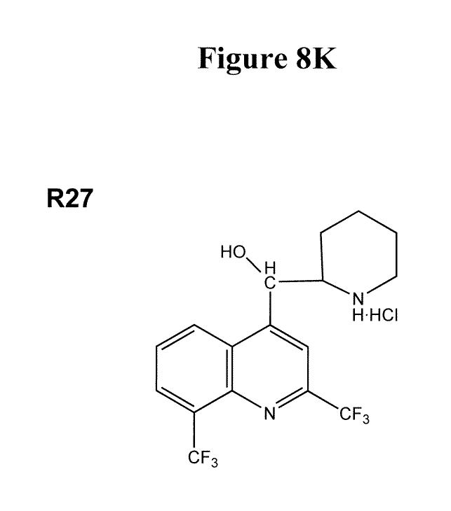

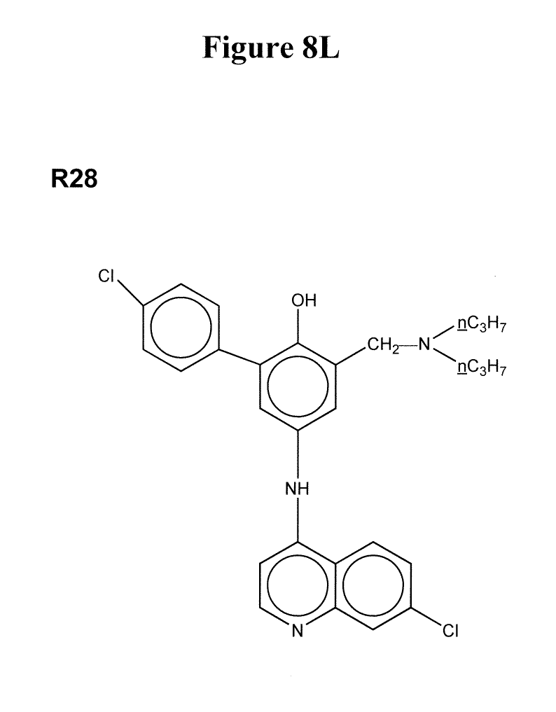

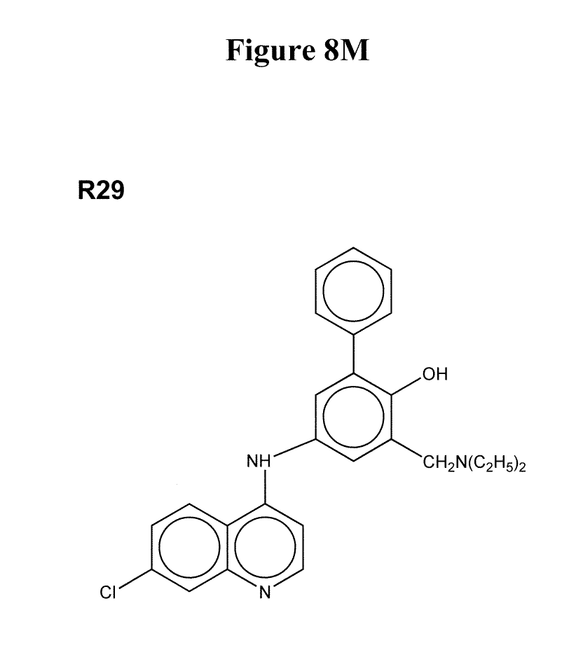

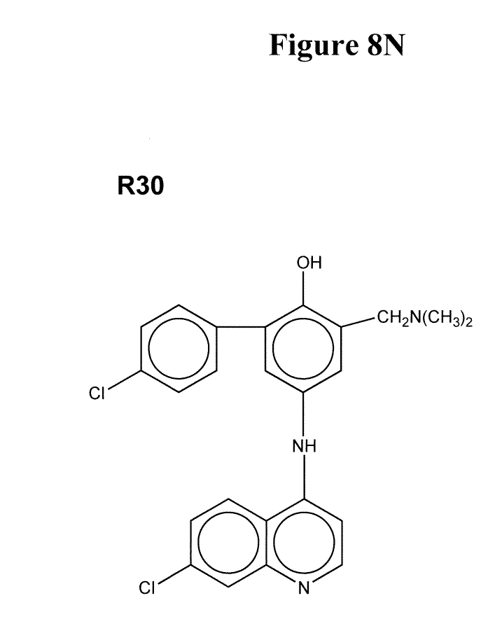

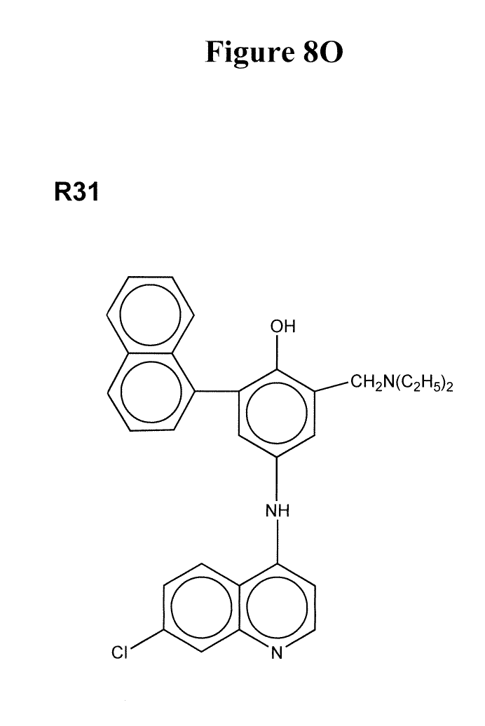

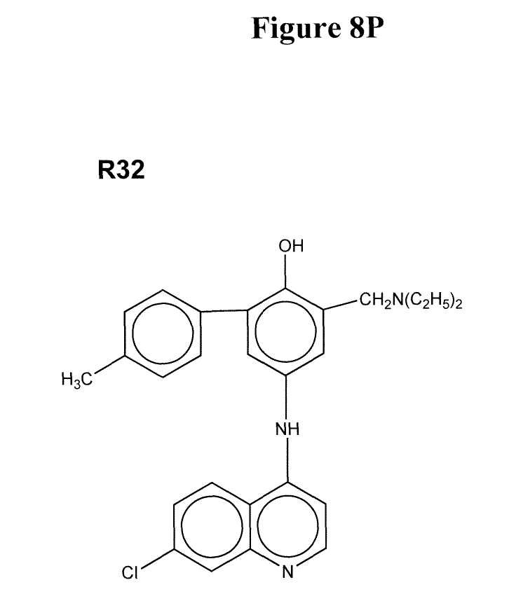

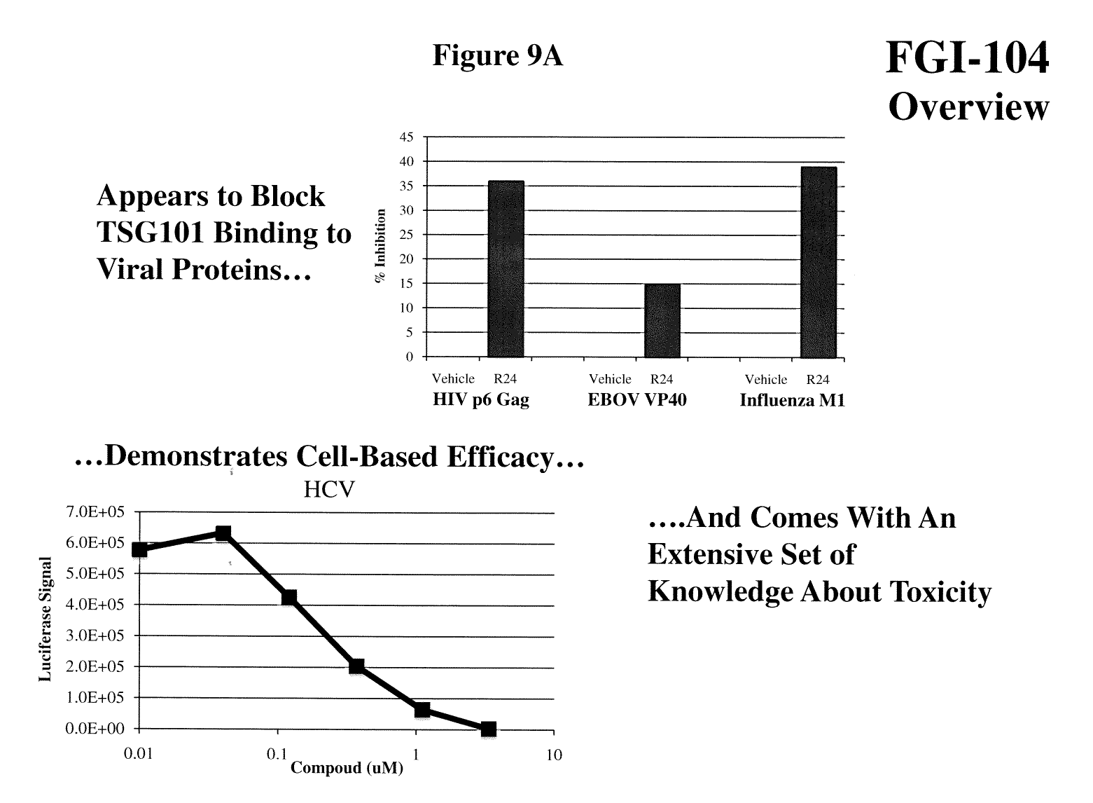

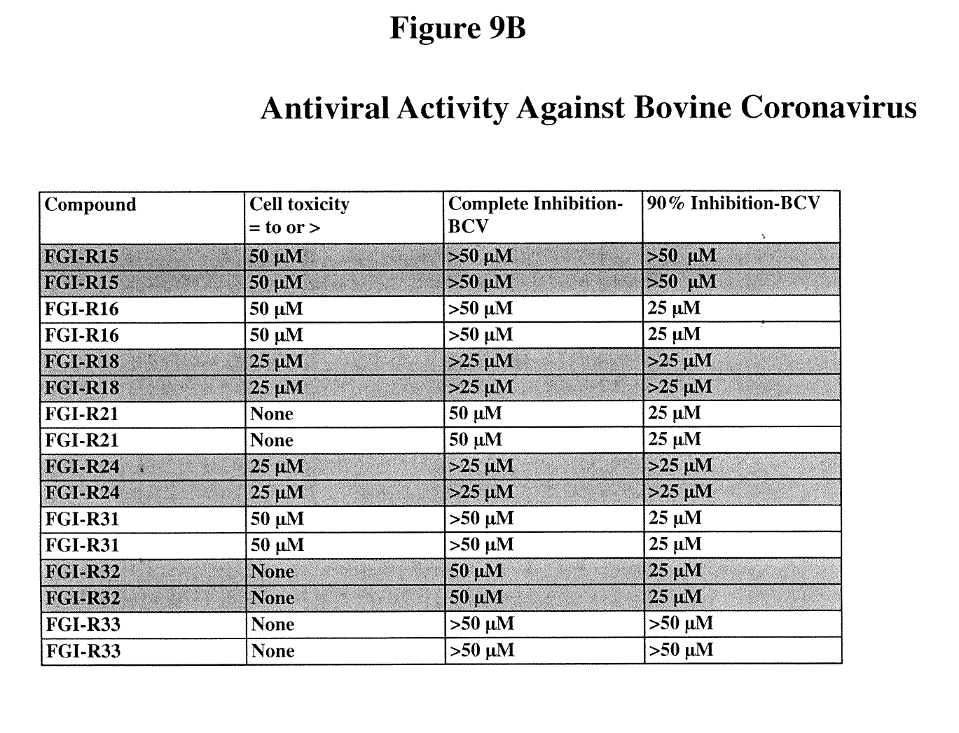

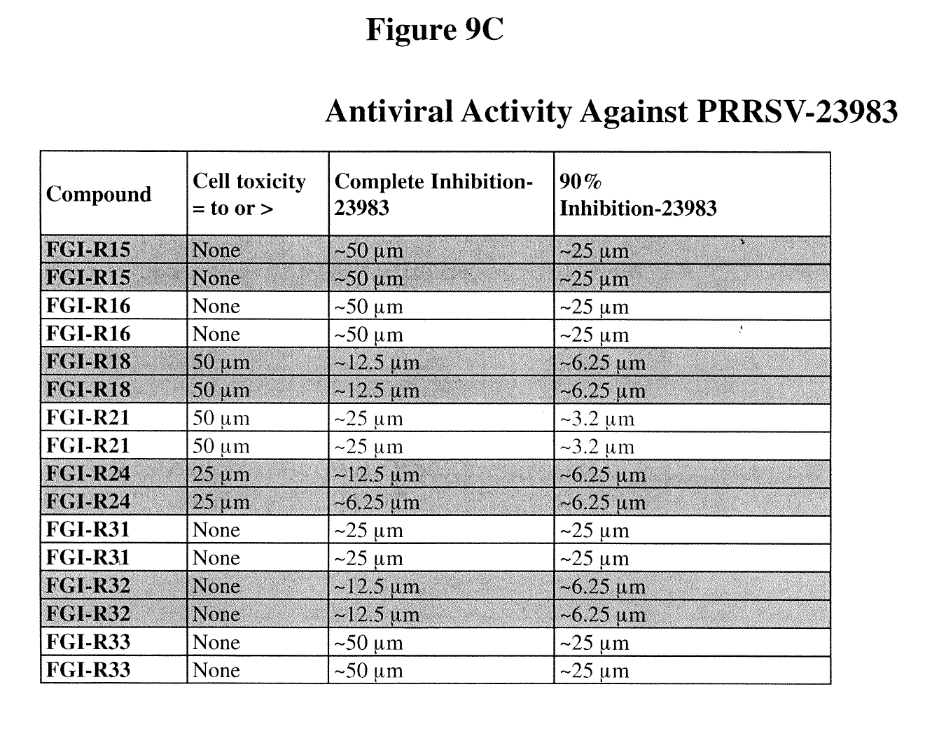

The compound design resulted in a family of compounds tested against a variety of viruses. Two of the most active and safest compounds are set forth in FIGS. 1 and 2, and are referred to herein as R19 and R24. These are not the only compounds of the FGI-104 family that are active, however. Out of a panel of potential active compounds, many have been tested and shown to have activity in inhibiting viral infection. Sixteen of the tested compounds are reflected in FIGS. 8A-8P. Although this is not all the active compounds, in fact all of these compounds were shown to be active, in testing (cell based assays) against two widely distributed and active viruses that impact commercial animal stocks—Bovine Corona virus and Porcine Reproductive and Respiratory Syndrome virus. The tables of activity for these compounds are set forth in FIGS. 9B and 9C, while an overview of the general character of FGI-104 activity against viruses is set forth in FIG. 9A.

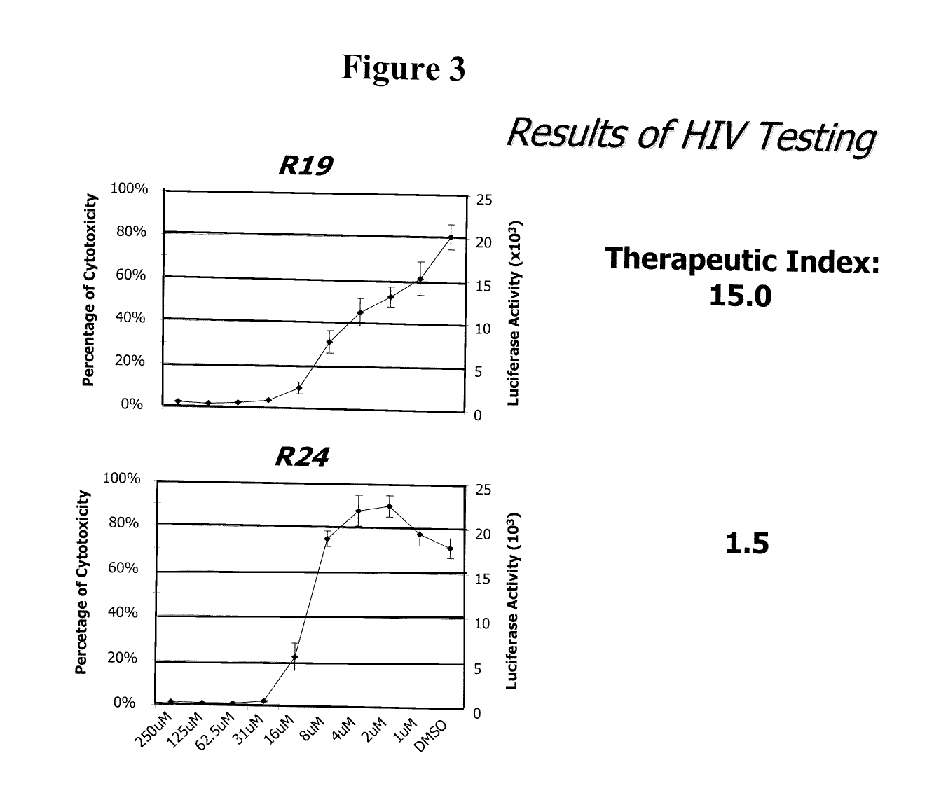

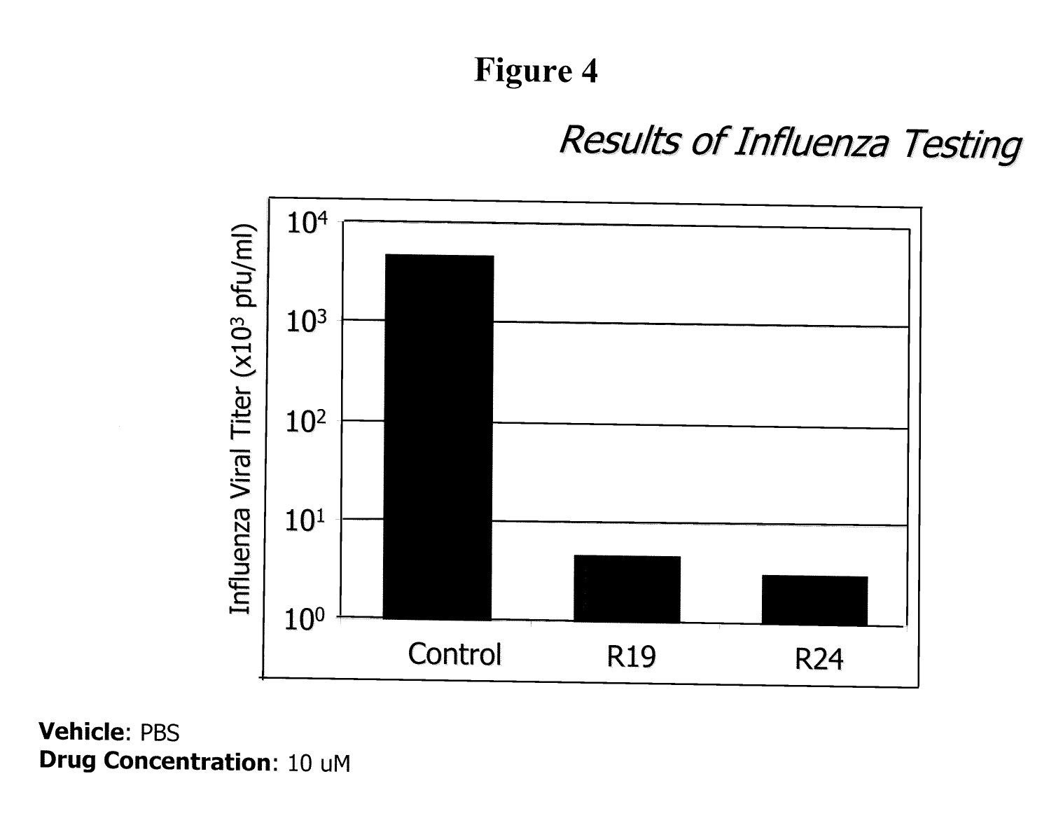

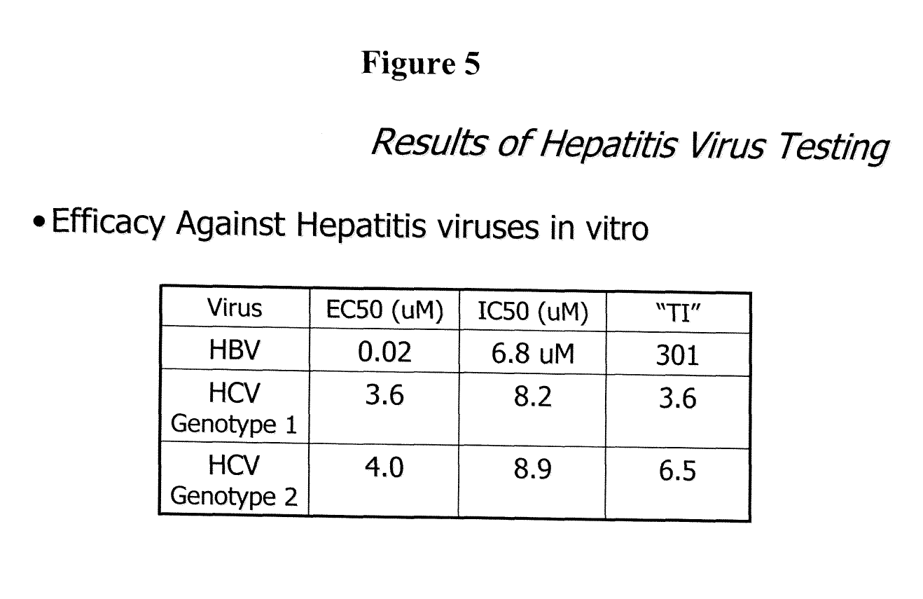

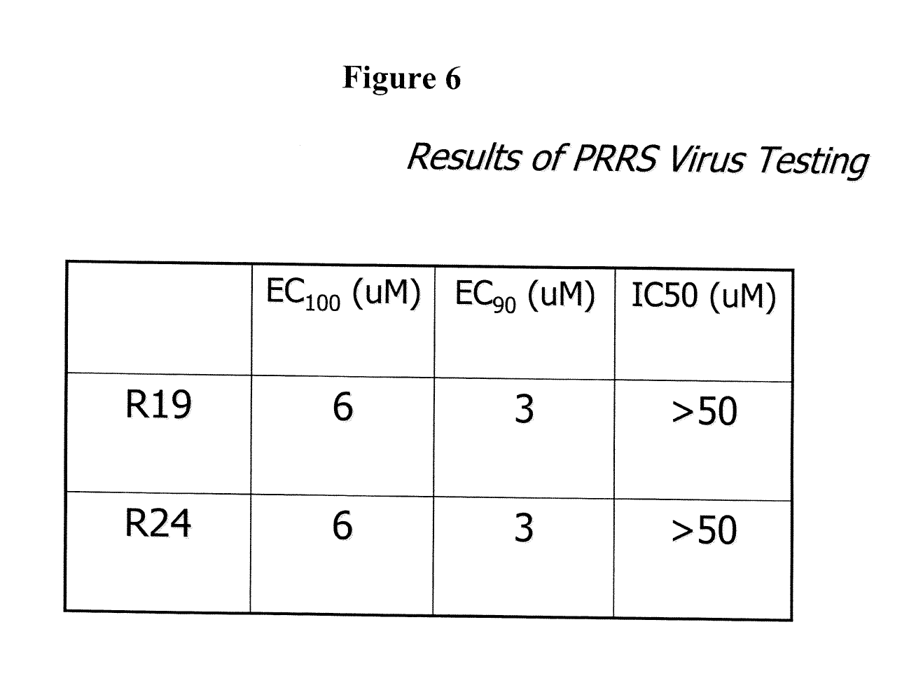

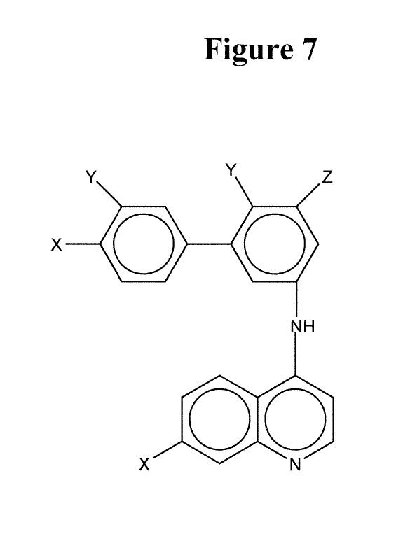

As noted, two of the most promising compounds, R19 and R24, have been shown effective in a wider variety of tests. Thus, FIG. 3 demonstrates the effectiveness of these two compounds in the inhibition, in a dose dependent relationship, of one of the most thoroughly researched of viral diseases, HIV. (Activity is shown as the amount of luciferase detected. In the absence of luciferase signal, there is no viral activity). Both of these compounds, at relatively mild dosages, showed complete inhibition of HIV. These same compounds have been shown to be effective in providing protection against infection challenge in in vitro studies against a wide variety of viral agents, including HIV, influenza, HBV, HCV, Punta Toro virus, and PRRS virus as shown in FIGS. 3-6. The design of the compounds of FGI-104 allows the provision of a neat compound structure for which activity can be provided. As is always the case, given the variation in species, viruses and individuals, not all FGI-104 compounds will show the same degree of activity in inhibiting all viruses in all mammalian species. Given the data provided, however, one can reasonably expect a compound drawn from the general formula of FIG. 7 will provide a mammalian host at least some protection against viral infection at a dosage value of 1 ng-250 mg/kg of host body weight. Within that range, those of skill in the art are well acquainted with titration analysis to arrive at an optimum dosage given a specific host and virus. See, e.g., “Remington: The Science and Practice of Pharmacy,” University of the Sciences in Philadelphia, 21st ed., Mack Publishing Co., (2005), the disclosure of which is hereby incorporated by reference in its entirety.

As noted, however, the invention of this application is not limited to the inhibition of viral infections in human, nor to any specific compound. Two viruses that infest commercial mammals are PRRS Virus, and Bovine Corona virus, which devastate pig and cow populations around the world. The pig is perhaps the world's most important non-human commercial mammal. The effectiveness of sixteen of the tested FGI-104 compounds against PRRS Virus, Bovine Corona virus, the testing being set forth in FIGS. 3-6 and 9, allows the identification of a variety of viruses against which the FGI-104 compounds may be expected to demonstrate inhibition. This list is not exhaustive, and to date, no virus has been identified which escapes the effects of all members of the FGI-104 family. But among prominent viruses which are important either from a human or animal health viewpoint are those such as influenza and PRRS, and viruses which present bioterrorism threats, such as Ebola, Marburg and other hemorrhagic fever viruses.

Among the multiple viruses whose infection may be treated by administration of FGI-104 compounds are certain families of viruses, including Group IV viruses (as demonstrated by activity against hepatitis C viruses); Group V viruses (as demonstrated by activity against influenza viruses); Group VI (represented by HIV virus) and Group VII (as demonstrated by activity against hepatitis B viruses).

One can predict that these compounds will have activity against other groups of viruses based on the broad-spectrum activity associated with targeting of the host. These would include viruses in Groups I, II, and III. The grouping of viruses, or viral families, is discussed below.

Viral Groupings:

Group I: viruses possess double-stranded DNA and include such virus families as Herpesviridae (examples like HSV1 (oral herpes), HSV2 (genital herpes), VZV (chickenpox), EBV (Epstein-Barr virus), CMV (Cytomegalovirus), Poxyiridae (smallpox) and many tailed bacteriophages. The mimivirus was also placed into this group.

Group II: viruses possess single-stranded DNA and include such virus families as Parvoviridae and the important bacteriophage M13.

| | | Virion- | | Type of |
|---|---|---|---|---|
| | | naked/ | Capsid | nucleic |
| Virus Family | Virus Genus | enveloped | Symmetry | acid |
| 1. Adenoviridae | Adenovirus | Naked | Icosahedral | ds |
| 2. Papovaviridae | Papillomavirus | Naked | Icosahedral | ds |
| | | | | circular |
| 3. Parvoviridae | B 19 virus | Naked | Icosahedral | ss |
| 4. Herpesviridae | Herpes Simplex | Enveloped | Icosahedral | ds |
| | Virus, Varicella | | | |
| | zoster virus, | | | |
| | Cytomegalovirus, | | | |
| | Epstein Barr virus | | | |
| 5. Poxviridae | Small pox virus, | Complex | Complex | ds |
| | Vaccinia virus | coats | | |
| 6. Hepadnaviridae | Hepatitis B virus | Enveloped | Icosahedral | ds |
| | | | | circular |
| 7. Polyomaviridae | Polyoma virus | ? | ? | ds |
| | (progressive | | | |
| | multifocal | | | |
| | leucoencephalo- | | | |
| | pathy) | | | |

### RNA Viruses

Group III: viruses possess double-stranded RNA genomes, e.g. rotavirus. These genomes are always segmented.

Group IV: viruses possess positive-sense single-stranded RNA genomes. Many well known viruses are found in this group, including the picornaviruses (which is a family of viruses that includes well-known viruses like Hepatitis A virus, enteroviruses, rhinoviruses, poliovirus, and foot-and-mouth virus), SARS virus, hepatitis C virus, yellow fever virus, and rubella virus.

Group V: viruses possess negative-sense single-stranded RNA genomes. The deadly Ebola and Marburg viruses are well known members of this group, along with influenza virus, measles, mumps and rabies.

| | | | | Type of |
|---|---|---|---|---|
| | | Virion-naked/ | Capsid | nucleic |
| Virus Family | Virus Genera | enveloped | Symmetry | acid |
| 1. Reoviridae | Reovirus, Rotavirus | Naked | Icosahedral | ds |
| 2. Picornaviridae | Enterovirus, Rhinovirus, | Naked | Icosahedral | ss |
| | Hepatovirus, Cardiovirus, | | | |
| | Aphthovirus, | | | |
| | Parechovirus, Erbovirus, | | | |
| | Kobuvirus, Teschovirus | | | |
| 3. Caliciviridae | Norwalk virus, Hepatitis | Naked | Icosahedral | ss |
| | E virus | | | |
| 4. Togaviridae | Rubella virus | Enveloped | Icosahedral | ss |
| 5. Arenaviridae | Lymphocytic | Enveloped | Complex | ss |
| | choriomeningitis virus | | | |
| 6. Retroviridae | HIV-1, HIV-2, HTLV-I | Enveloped | Complex | ss |
| 7. Flaviviridae | Dengue virus, Hepatitis C | Enveloped | Complex | ss |
| | virus, Yellow fever virus | | | |
| 8. Orthomyxoviridae | Influenzavirus A, | Enveloped | Helical | ss |
| | Influenzavirus B, | | | |
| | Influenzavirus C, | | | |
| | Isavirus, Thogotovirus | | | |
| 9. Paramyxoviridae | Measles virus, Mumps | Enveloped | Helical | ss |
| | virus, Respiratory | | | |
| | syncytial virus | | | |
| 10. Bunyaviridae | California encephalitis | Enveloped | Helical | ss |
| | virus, Hantavirus | | | |
| 11. Rhabdoviridae | Rabies virus | Enveloped | Helical | ss |
| 12. Filoviridae | Ebola virus, Marburg | Enveloped | Helical | ss |
| | virus | | | |
| 13. Coronaviridae | Corona virus | Enveloped | Complex | ss |
| 14. Astroviridae | Astrovirus | Naked | Icosahedral | ss |
| 15. Bornaviridae | Borna disease virus | Enveloped | Helical | ss |

### Reverse Transcribing Viruses

Group VI: viruses possess single-stranded RNA genomes and replicate using reverse transcriptase. The retroviruses are included in this group, of which HIV is a member.

Group VII: viruses possess double-stranded DNA genomes and replicate using reverse transcriptase. The hepatitis B virus can be found in this group.

The viruses discussed above are grouped largely in terms of human infection. As noted above, the FGI-104 compounds are effective in disrupting or interfering with the host mechanisms necessary for viral propagation that are highly conserved among mammalian or eukaryotic species. Consequently, these compounds could have application for human or veterinary viral diseases. These viral diseases could include but are not limited to PRRS virus, porcine or bovine circoviruses, porcine or bovine corona viruses, porcine or bovine RSV, porcine or bovine influenza, EIAV, bluetongue, or foot and mouth disease (FMD) viruses.

Some viruses are causative of more chronic diseases and the morbidity or mortality relates to the presence of virus. These diseases include hepatocellular carcinoma (associated with either HBV or HCV), chronic fatigue syndrome (associated with EBV) and other diseases linked with viral infection. As the compounds of the FGI-104 family have proven effective in inhibiting or treating these viruses, the administration of these compounds, in vivo, should provide a method of controlling and relieving these chronic conditions and the associated morbidity.

The compounds above could be used for the treatment or prevention (prophylaxis) of single viral pathogens (e.g., HIV or HBV) or combinations thereof (HIV and HBV). Likewise, these individual or broad-spectrum applications could entail any or all of the virus groups detailed above.

Another method could be the use of the compounds for certain indications associated with one or more viruses. For example, these compounds could be used for the prevention or treatment of respiratory virus infections, which can be caused by one or more of the pathogens from the groups identified above. Likewise, these compounds could have application against one or more blood-borne pathogens (e.g., HIV and/or HBV and HCV).

The compounds could have application for the prevention, treatment, or maintenance of acute or chronic viruses. Acute applications include short-term prevention or treatment of viral infection, examples of which include influenza, rotavirus or filovirus infection. Chronic applications could include recurrent outbreaks, (such as is observed with genital herpes) or infrequent outbreaks (such as those associated with zoster infection during shingles). Likewise, treatment could be intended over the long term to maintain low levels of viral load for chronic virus infection (e.g., for HIV, HBV or HCV treatment).

“Treatment” in the context of this application for patent, and this invention, embraces both prophylaxis and therapeutic administration. Administration of the FGI-104 compounds at or before the “challenge” of a virus should provide a means of inhibiting or reducing infection in those likely to encounter the virus, such as service people or others dispatched to areas where viruses are found against which they might have little or no natural resistance, such as Ebola virus. Treatment can be after infection. Indeed, research suggests that days after infection, administration of FGI-104 compounds may be effective in arresting and/or reversing the course of viral infection. Treatment also embraces extending the survivability of the infected subject, so that the body's natural defense mechanisms can combat and overwhelm the viral infection, and reducing the level of viral infection.

The compounds could be used alone or in combination with the current standards of care for any of the viruses indicated above. In general, although other modes of administration are contemplated, oral, cutaneous, subcutaneous, suppository, IV or IM injection, or sustained IV administration, are preferred routes. Dosages will vary from mammal to mammal and virus to virus. As a general range, 0.001 mg/kilo/day-200 mg/kilo/day, IV, are target dosages. To this end, the FGI-104 compounds have been demonstrated to exhibit robust activity in animal models of otherwise deadly viruses (Ebola, Marburg) that doses in the range of 0.1-10 mg/kg, delivered once pre- or post-infection, are sufficient to prevent virus-mediated death. Those of skill in the art are well equipped by conventional protocols, given the identification of targets and compounds herein, to identify specific dosages for specific mammals, specific viruses, and specific modes of administration.

As indicated, the specifically identified compounds, R19 and R24, are representative only. Variations and derivations of the core parental compounds are one aspect of the invention. For example, an oxime or methoxyamine derivative of the parent compound could provide an opportunity for oral delivery. The advantages of oral delivery can include ease of administration, patient compliance and/or distribution and reimbursement. A representative core structure, with substituents, embracing the most active compounds, is set forth in FIG. 7. Each substituent X is independently H or an electron donating group, which may be selected from the group including chloro or other halogen, alkoxy (—OR), hydroxyl (—OH); aryloxy (—OAr), trialkylammonium (—NR₃+), alkylamido (—NHCOR, —NRCOR′), arylamido (—NHCOAr, —NRCOAr, —NArCOAr), arylcarbamoyl (—NHCOOAr, —NRCOOAr), alkylcarbamoyl (—NHCOOR, —NRCOOR′), cyano (—CN), nitro (—NO₂), ester (—COOR, —COOAr), or alkyl halo. Each substituent Y is independently H, alkyl of 1-4 carbon atoms, hydroxy, alkoxy or methylene. Substituent Z is a di-or-tri akly amino, or alkyl di or tri amino, optionally substituted with a halogen moiety.

### R24—A Representative Study

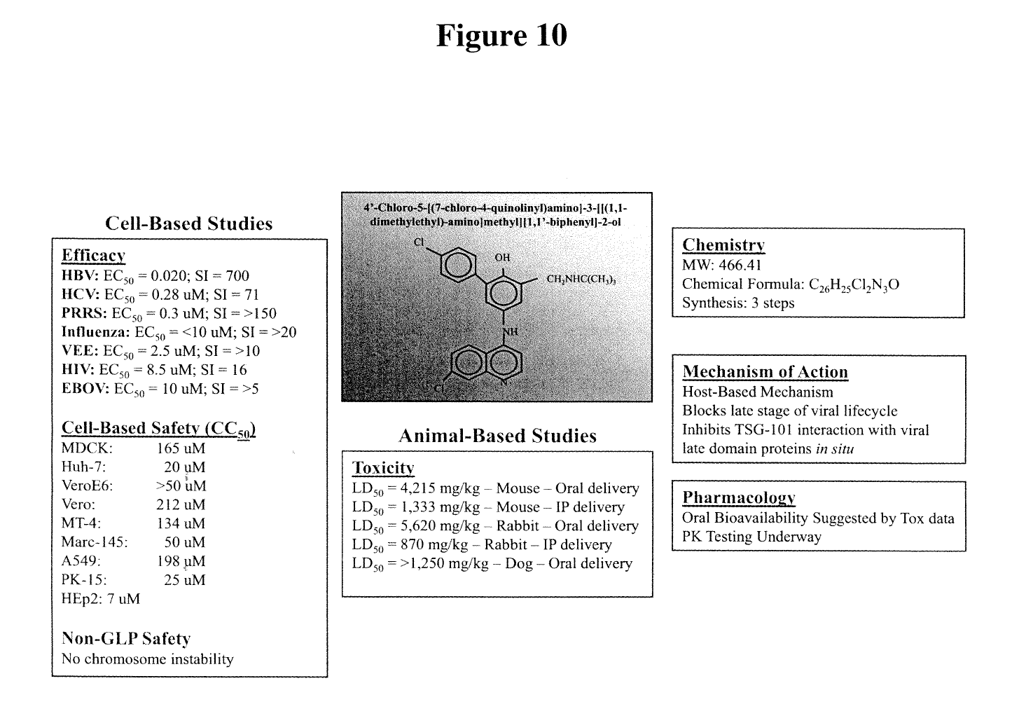

The FGI-104 family of compounds, even the more narrowly circumscribed family of compounds embraced by the core structure of FIG. 7, includes a large number of variations. Different compounds will have different activities against different viruses, as reflected by the data set forth in FIGS. 9A-9C. It is not practical or desirable to test all active compounds of this family against all viruses, or even a sampling. As noted however, Compound R24, 4′chloro-[7-Chloro-4-quinolinyl)amino]-3-[(1,1-dimethyl,ethyl)amino]methyl][1,1′-biphenyl]-2-ol, generally isolated as a hydrochloride salt, was the subject of non-public testing in the 1980s as a possible treatment for malaria. This data, not generally publicly available, establishes this compound as safe and non-toxic, in a variety of mammals, and mammalian models for humans, through diverse delivery routes. Of particular moment, as reflected in FIG. 10, is the fact that this compound is effective administered orally. Where multiple administration of compounds is required, as may be practiced in the claimed invention, follow-up and completion of the course of administration is notoriously poor where injection is required. In addition to the other routes given above, oral administration gives the advantage of easier compliance.

The wealth of data on R24 makes it a suitable representative target. It is known to be well tolerated and have a large safety, low toxicity value. It exhibits safety, again as shown in FIG. 10, in a wide variety of cell based assays. It was shown not to induce significant chromosomal instability over a 28 day course of administration. And it has shown efficacy against a wide variety of viruses, including Hepatitis B and Hepatitis C, PRRS, Influenza, VEE, HIV and Ebola, each of which bears strong resemblance to other viruses amenable to treatment with FGI-104 compounds. Accordingly, this particular compound is singled out for further study, as an example of the anti-viral activity exhibited by this class of compounds.

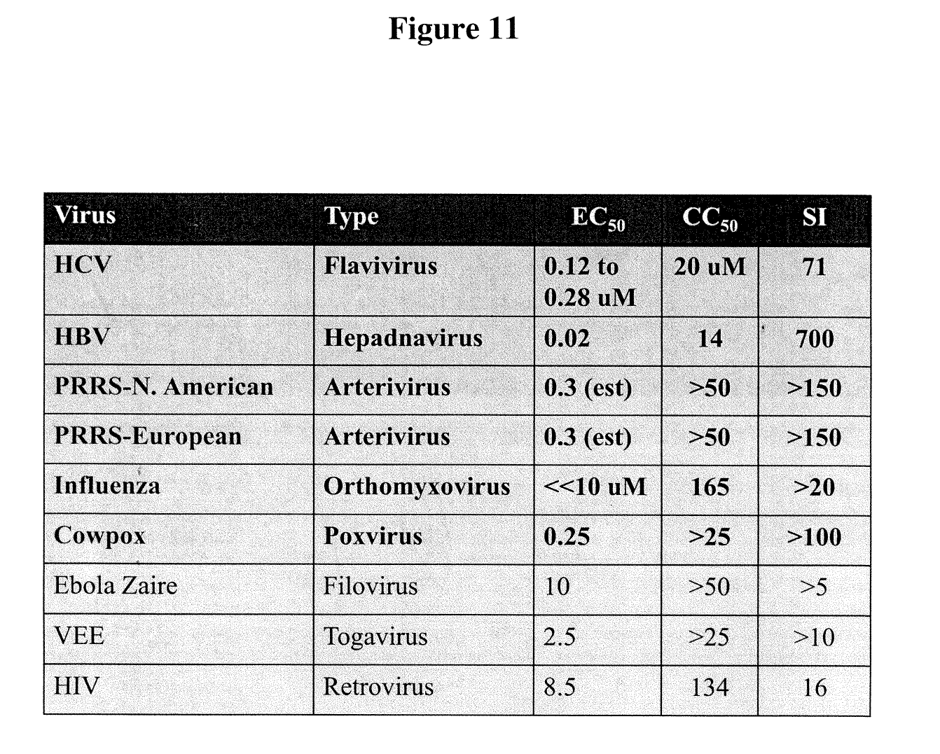

In addition to the capsule information provided in FIG. 10, the breadth of activity for R24 is summarized in Table form in FIG. 11. Again, the compound has shown activity across a broad selection of families of viruses—underscoring the fact that these compounds act to inhibit a host protein, rather than trying to attack the wide variety of different viral proteins. This is not only valuable in providing pan-viral activity, but in reducing the pressure on the virus to mutate and thereby escape the efficacy of the treatment.

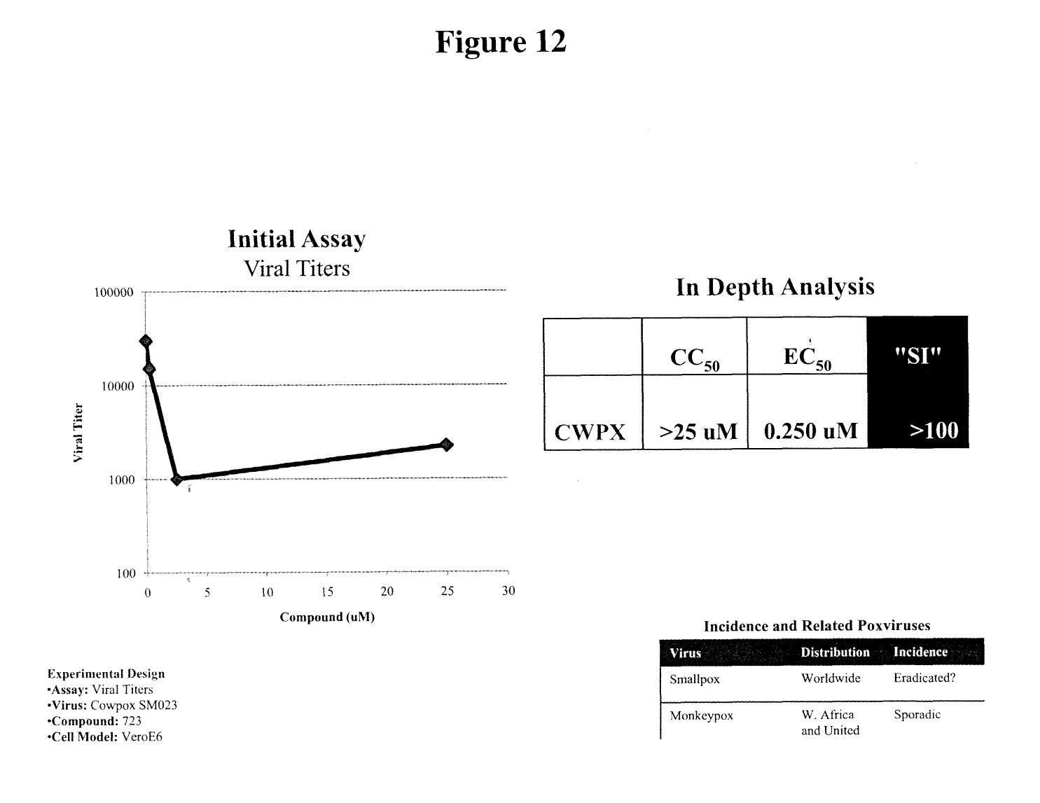

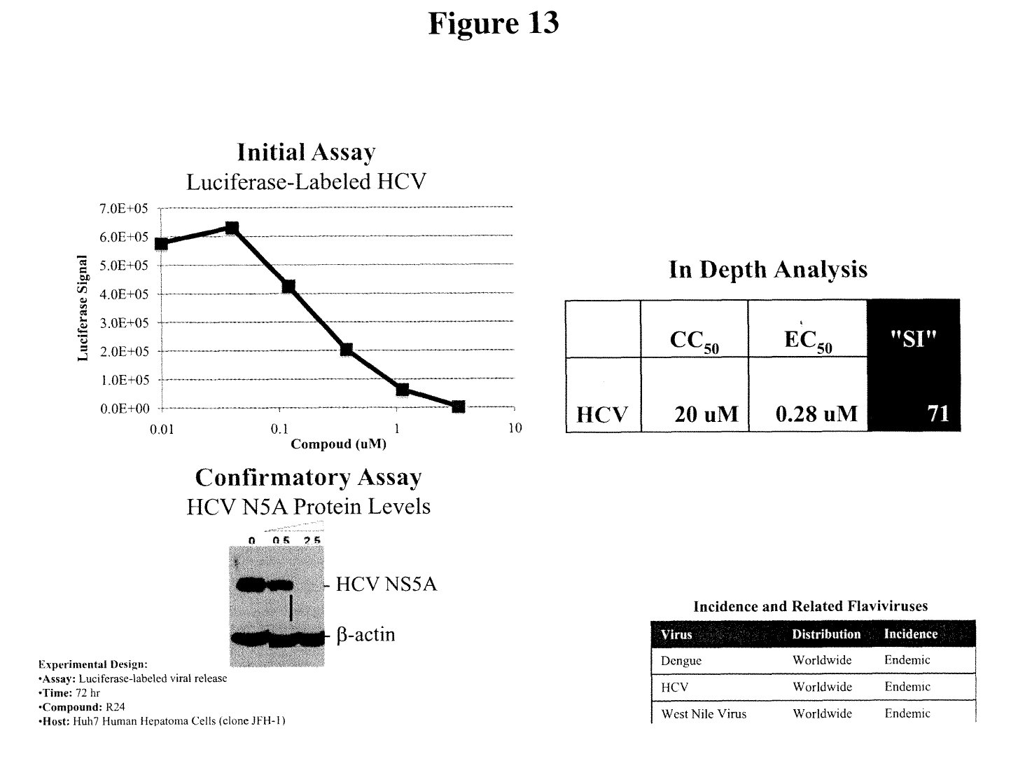

Representative testing for the anti-viral R24 is depicted by both graph and table in FIG. 12, where the inhibition of cowpox by R24 is given. In the cell assay against Vero cells provided, administration of R24 to the infected cell culture dropped viral titer dramatically, at low dosages, giving an EC₅₀ value of 0.250 uM, with a large CC₅₀ value of greater than 25 uM. This gives a Safety Index, or SI, of more than 100, indicating that the compound is both safe and effective.

Effectiveness against HCV is demonstrated by a luciferase assay, where the virus is luciferase tagged, or labeled. As shown in FIG. 13, in a measured dose response fashion, delivery of R24 to the infected cells (Huh 7 Human Hepatoma Cells) extinguished the luciferase signal, demonstrating 100% inhibition of the virus. Again, the relative values for CC₅₀ and EC₅₀ give this compound a large Safety Index of 71, For certainty, protein blotting assays were conducted. At 2.5 uM, the “signal protein” N5A of HCV was completely missing (β-actin, a positive control, was detected) confirming that viral infection and replication had been blocked. As shown in FIG. 13, this virus is closely linked to other known inimical viruses, both in terms of human illness, and as bioterrorism weapons, Dengue Fever and West Nile Virus, all treatable with the same FGI-104 compounds.

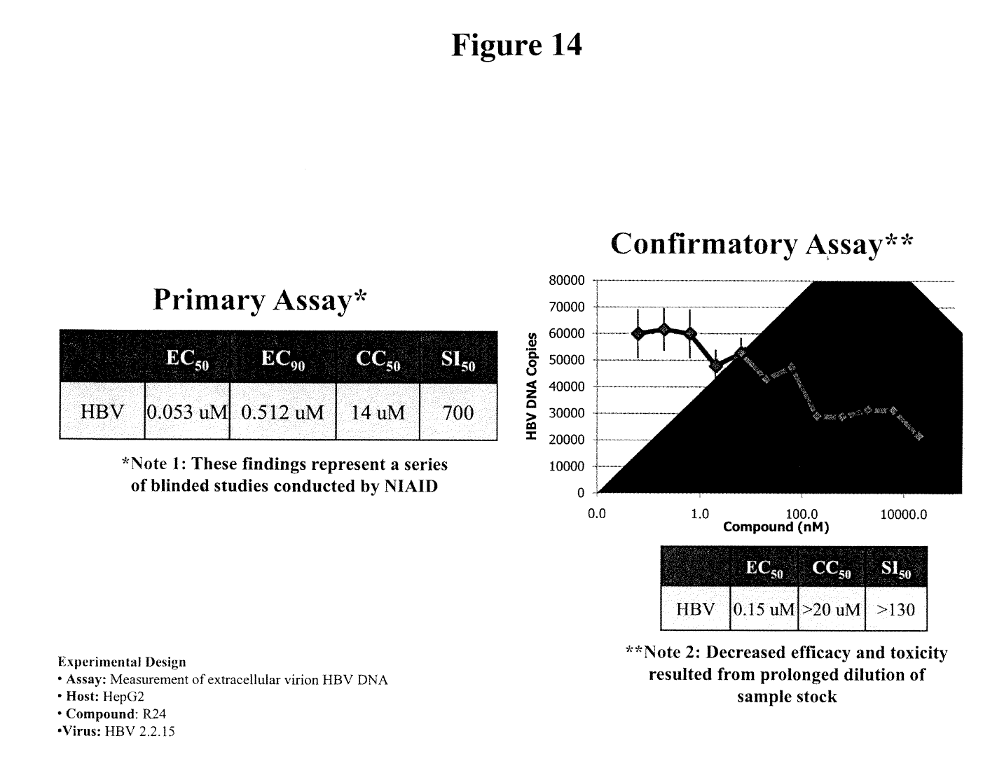

In addition to Hepatitis C, R24 is effective against HBV, as set forth in FIG. 14. Although dilution of the sample stock “foxed” actual stock, giving a relatively falsely low but comfortable Safety Index of more than 130, the primary assay run for R24 against HBV using HepG2 cells showed a high level of activity, as measured by virion DNA. These results were obtained from blinded studies conducted by the National Institute for Allergies and Infectious Diseases, further reaffirming the reliability and accuracy of the data presented in FIG. 14.

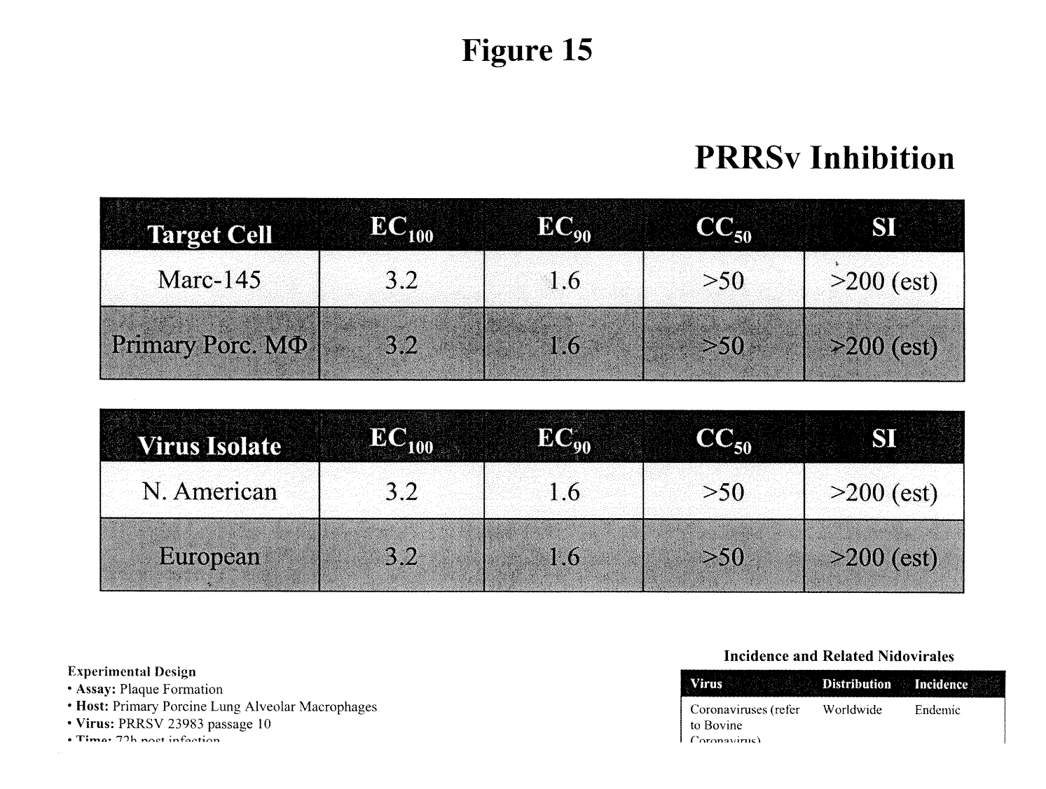

As noted above, important mammalian hosts treatable by this family of compounds include not only humans, but veterinarily and commercially important animals. Although monkeys, dogs, cats, mice, rats, horses, rabbits, cattle, sheep and goats are all important hosts to be treated for viral infection, pigs may be the dominant commercial animal, worldwide. The United States Department of Agriculture has characterized PRRS virus as a significant, worldwide, agricultural concern related to animal health and causes large economic losses to producers, and labeled a biosecurity threat. (Project 2008-2017). FIG. 15 demonstrates the effectiveness of R24 in inhibiting PRRS, where the host is the virus's natural target, primary porcine lung alveolar macrophages. It is important to note that the administration of R24 in the inhibition trials reflected in FIG. 15 is 72 hours post infection, demonstrating that the compounds have therapeutic, as well as earlier demonstrated prophylactic, effectiveness. The high Safety Index for this compound is a product of its spectacular effectiveness as reflected by the EC₉₀ value.

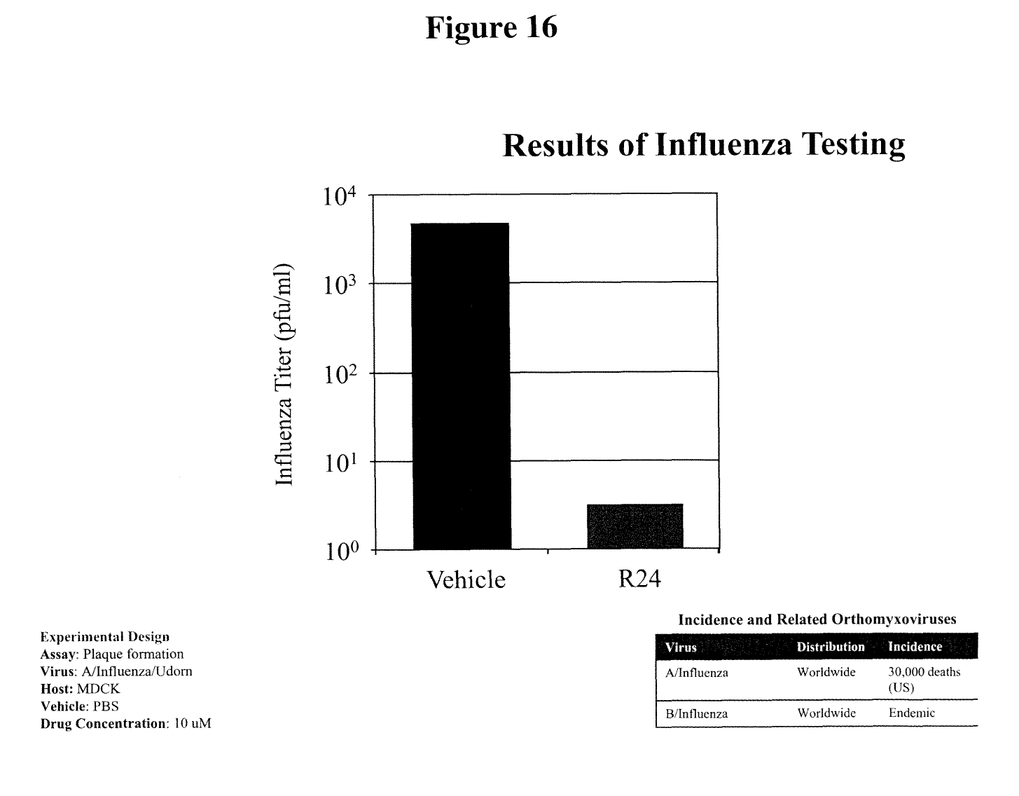

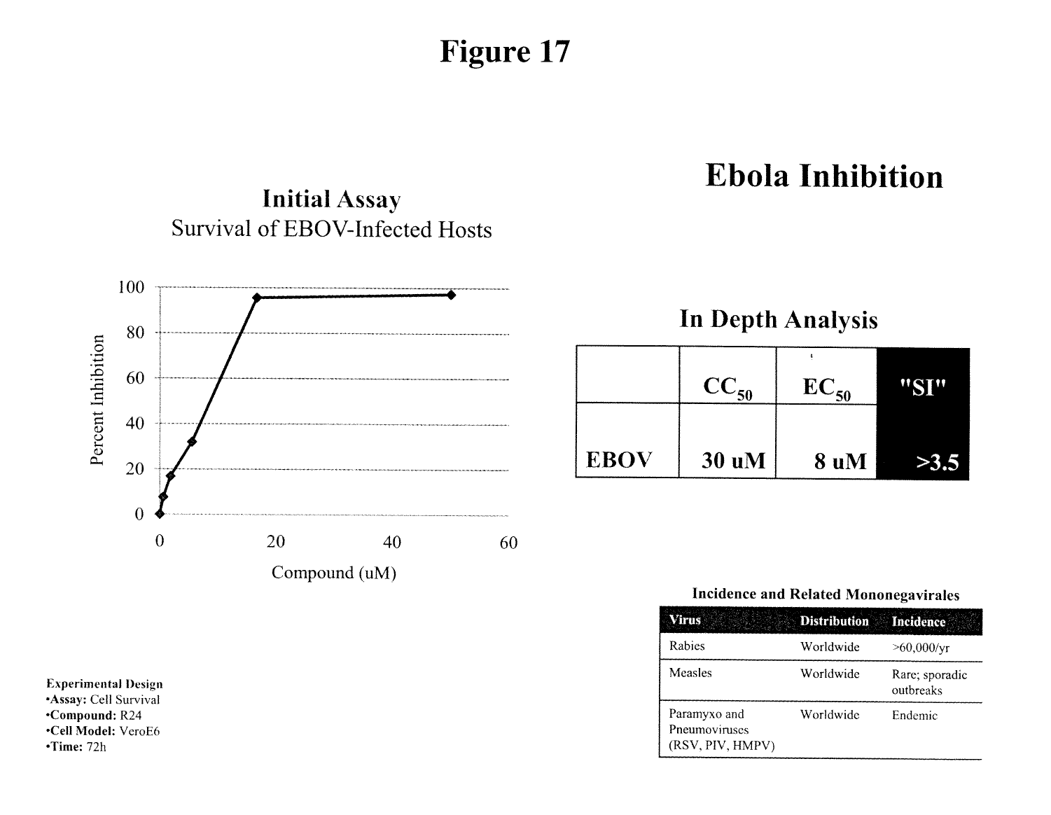

R24 is also effective against viruses that infect both animals and humans with wide activity. Influenza kills thirty thousand people in the United States every year, and is endemic worldwide. Complicating matters is the fact that various serotypes and strains of the virus often are not protected by a single vaccine. Those most in need of protection, the elderly and those immunologically challenged, frequently benefit least from the vaccine. As shown in FIG. 16, R24 and the other compounds of the FGI-104 family are remarkably effective in inhibiting influenza viral activity, this time in a MDCK cell-based assay. Clearly, by targeting interaction between a host protein, TSG101, and viruses in general, R24, and the FGI-104 compounds, are effective against a wide distribution of viruses, exhibiting potent activity with little safety risk.

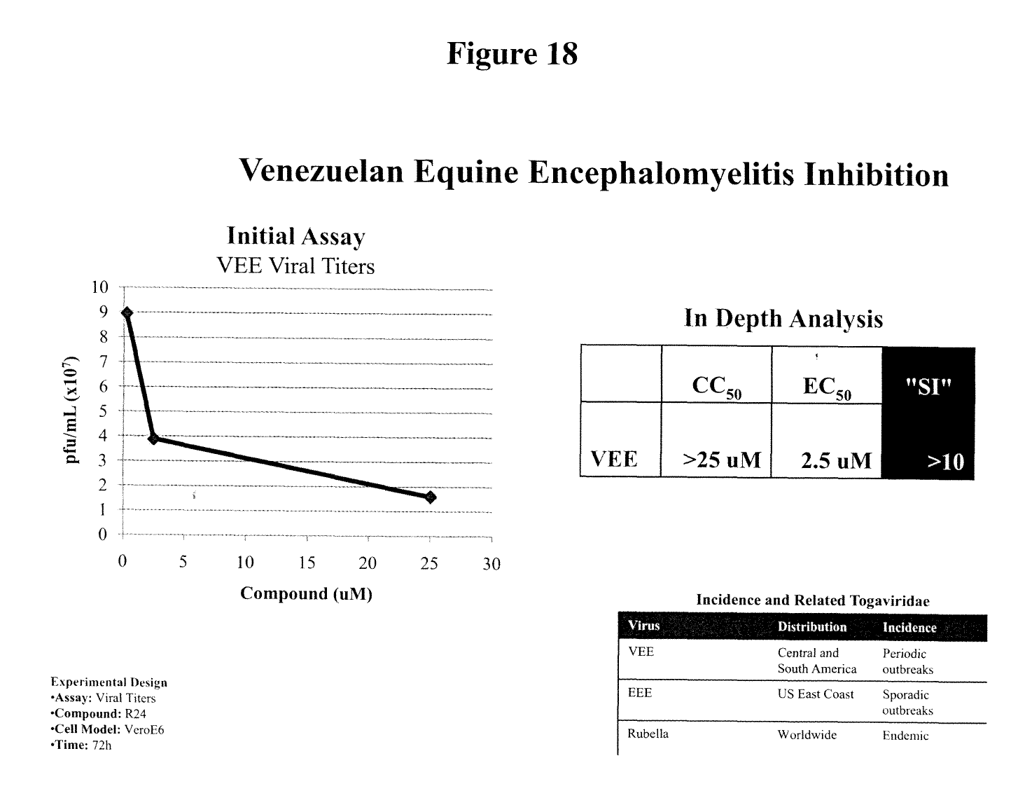

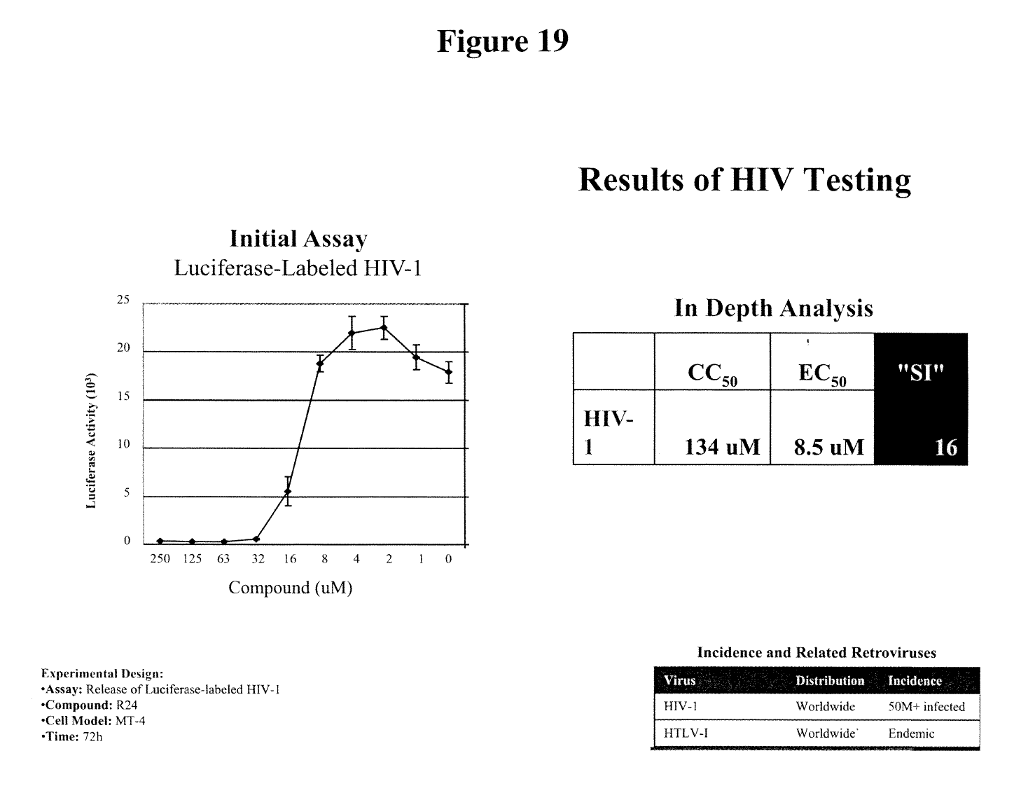

Alphaviruses, like Venezuelan Equine Encephalomyelitis, constitute yet another class of viruses that infect both humans and mammalian animals like horses and cows. In 1995, an outbreak in South America killed an estimated 20,000 people and huge numbers of animals. As shown in FIG. 18, R24 provided dramatic inhibition of VEE at levels of 25 uM, again administered 3 days post-infection. Among other viruses related to VEE, and another Togaviridae member, is rubella (German measles).

HIV has a different viral maturation cycle than most viruses. It has also proved adept in evading most agents, biological and chemical, targeted at the virus, or the effects of the virus, itself. More resources have been devoted to finding a treatment or preventive agent for HIV than any other virus. Yet, treatment of this virus remains elusive. In FIG. 19, the results of R24 administration to MT-4 cells infected with HIV are shown by graph and in table format. As shown (increasing dosage runs from right to left) in a dose response fashion, R24 inhibits HIV-1 activity, dropping the luciferase detection to effective zero at a relatively low dose, when administered 72 hours post infection (MT-4 cells). The compound is well tolerated, and gives a Safety Index of 16 with an EC₅₀ value of 8.5 uM.

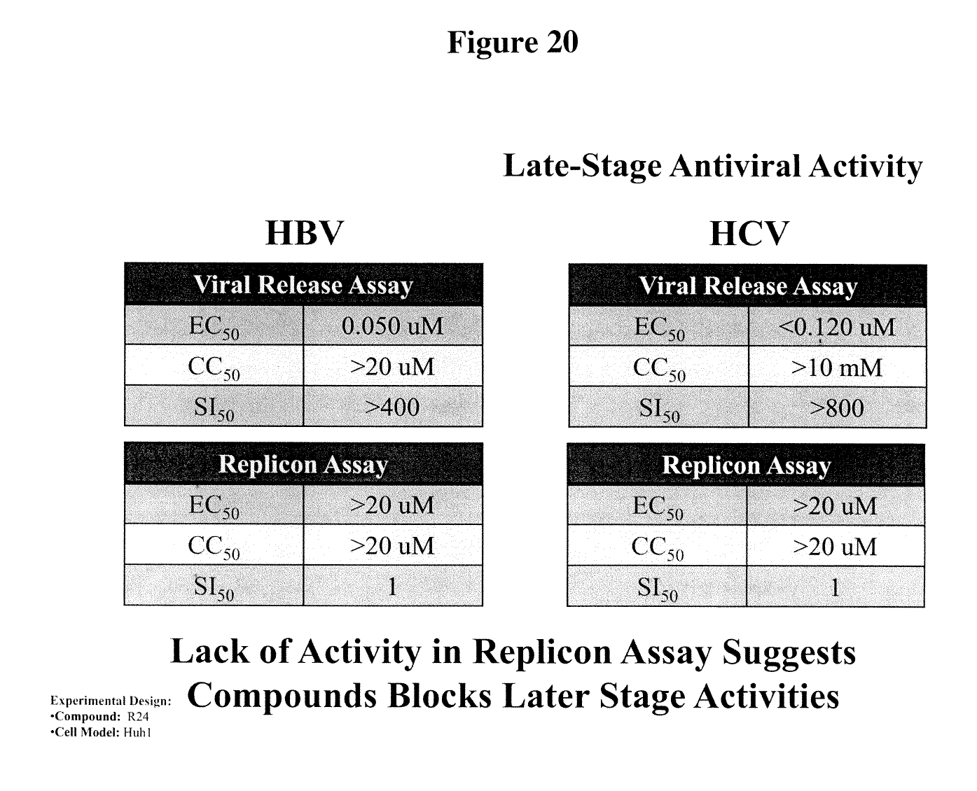

As discussed above, R24, like the family of FGI-104 compounds, was targeted to interfere with the interaction between viruses and TSG101, giving the compound a greater range of viral inhibition than most virus targeted agents. Confirmation that the agents work to interfere with, and thus treat, viral infection comes from the assays shown in FIG. 20, which contrasts the effectiveness in suppressing viral release of HBV and HCV from Huh1 cells, and replicon formation. As shown, R24 does little to inhibit replicon formation, but is effective in preventing viral release. This strongly suggests that the interaction or activity of R24 occurs after viral protein synthesis is complete (thus replicon formation) but before complete maturation and release. As TSG101 is implicated in “assisting” virus particles to egress to the cell surface for maturation, the activity observed is consistent with the understanding that the active agent, here R24, inhibits the interaction between the virus and TSG101. Similar results can be expected in treatment of other viruses, such as respiratory syncytial virus (RSV), parainfluenza virus (PIV) and Human metapneumovirus (HMPV).

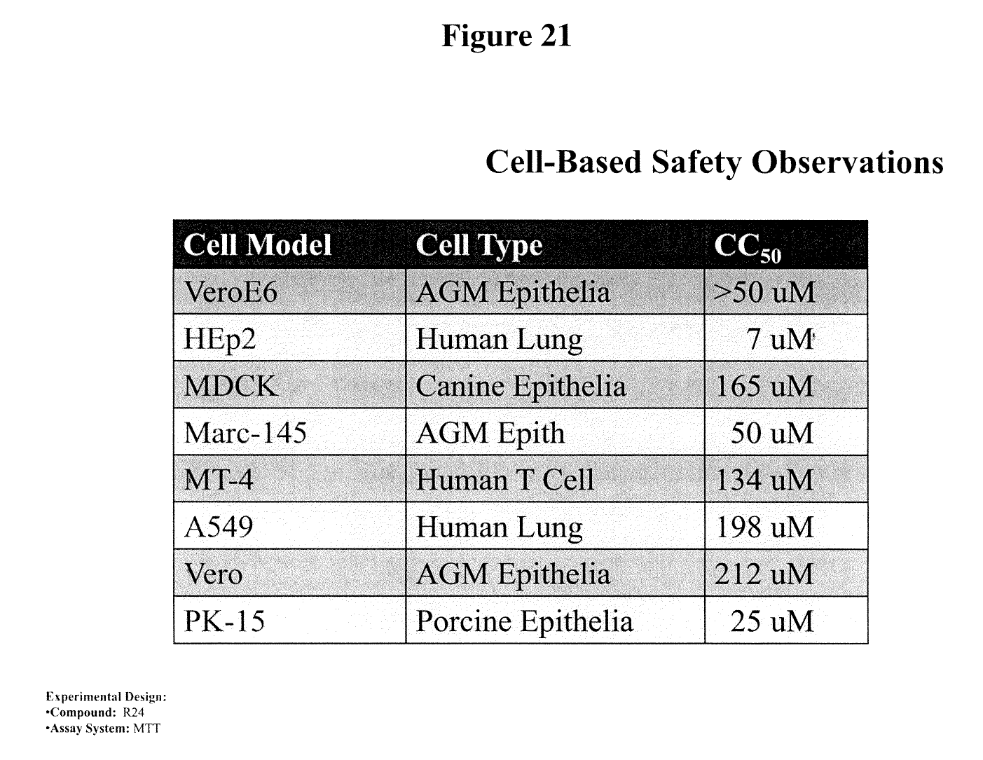

As noted previously, proprietary data demonstrates the safety of R24, and the FGI-104 compounds, for a variety of mammalian models. R24 has been tested in a variety of cell based assay systems, where safety has also been demonstrated. The cell models used and the corresponding CC₅₀ values obtained are presented in FIG. 21. Taken together with the animal models tested, a high degree of confidence in safety and lack of toxicity is demonstrated for this family of anti-viral compounds.

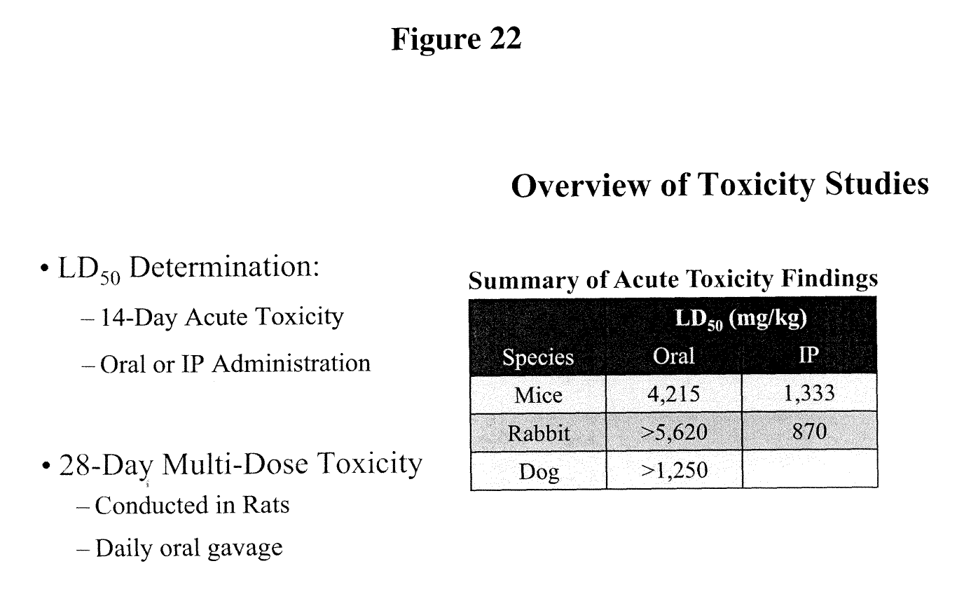

This lack of toxicity is summarized in FIG. 22, reflecting the results of testing on both 14 day acute toxicity trials, and 28-day multidose toxicity trials. Whether administered IP or orally, the compounds are well tolerated in mammalian models, and thus present the opportunity to treat viral infection in a clinical setting. Although not all compounds have been tested for all modes of administration, the data presented, together with standard assays, allow those of skill in the art to arrive at conclusive determinations as to effectiveness, toxicity, and administration protocol without undue experimentation.

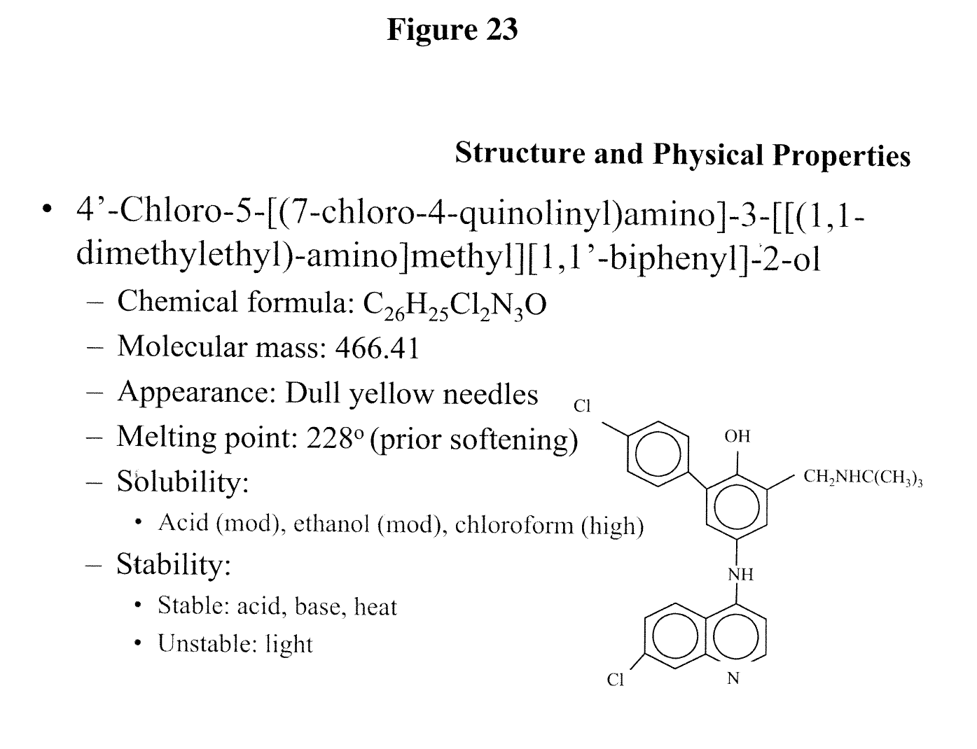

R24 is subject to straight forward synthesis. Specific important physical and chemical information is presented in FIG. 23. While the compound exhibits instability to light, R24 is easily protected from light, whether present as a solid, or prepared in a solution or suspension for administration. The range of carriers available, is therefore, quite large. As a consequence, the compositions and methods of this invention embrace the FGI-104 compounds as neat solids, as pharmaceutically suitable preparations, in a pharmaceutically acceptable carrier of a variety of types. Compositions of the present invention may further comprise a pharmaceutical composition comprising a therapeutically effective amount of any of the small molecules (or combinations of small molecules) described above together with other materials, such as a suitable carrier, excipients, etc., for administration to a human or animal experiencing a viral infection or at risk of a viral infection. Such pharmaceutical compositions may be in solid, gel or liquid form and may be administered as appropriate to an individual IV, IM, IP or parenterally, topically, subcutaneously, orally, or through mucosal surfaces and routes (including, for example, rectal and vaginal suppositories). The exact dosage corresponding to a therapeutically effective amount will vary from mammal to mammal and virus to virus. The dosage ranges set forth above in specific examples for each of the FGI-104 compounds tested are representative, and provide sufficient information to those of skill in the art, following the assay procedures set forth herein and known to those of skill in the art, to arrive at suitable dosage values for any given virus and mammalian host. Those of skill in the art are well equipped by conventional protocols, given the identification of targets and compounds herein, to identify specific dosages for specific mammals, specific viruses, and specific modes of administration. See, e.g., “Remington: The Science and Practice of Pharmacy,” University of the Sciences in Philadelphia, 21st ed., Mack Publishing Co., (2005), the disclosure of which is hereby incorporated by reference in its entirety. As noted, the FGI-104 compounds may be administered as the sole active agent administered, either prophylactically or therapeutically, or together with other active agents. Although the other active agents may be other anti-viral agents, it is envisaged that the FGI-104 compounds may be administered together with agents targeted at secondary effects of the viral infection or associated chronic diseases, or for example, an anti-bacterial agent.

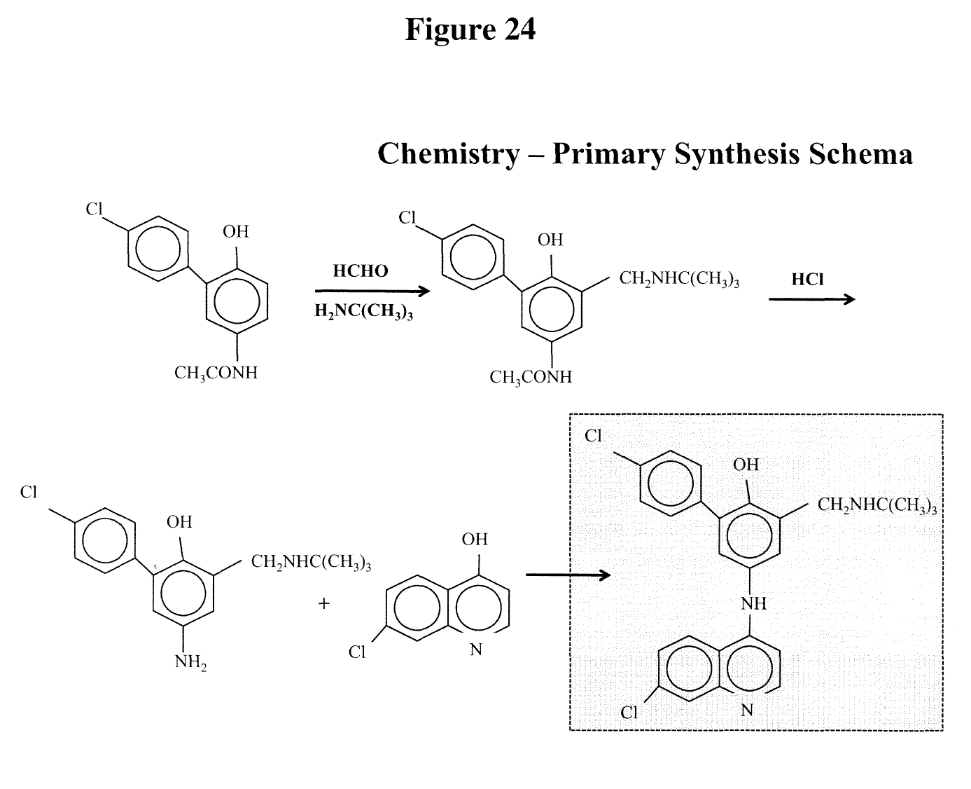

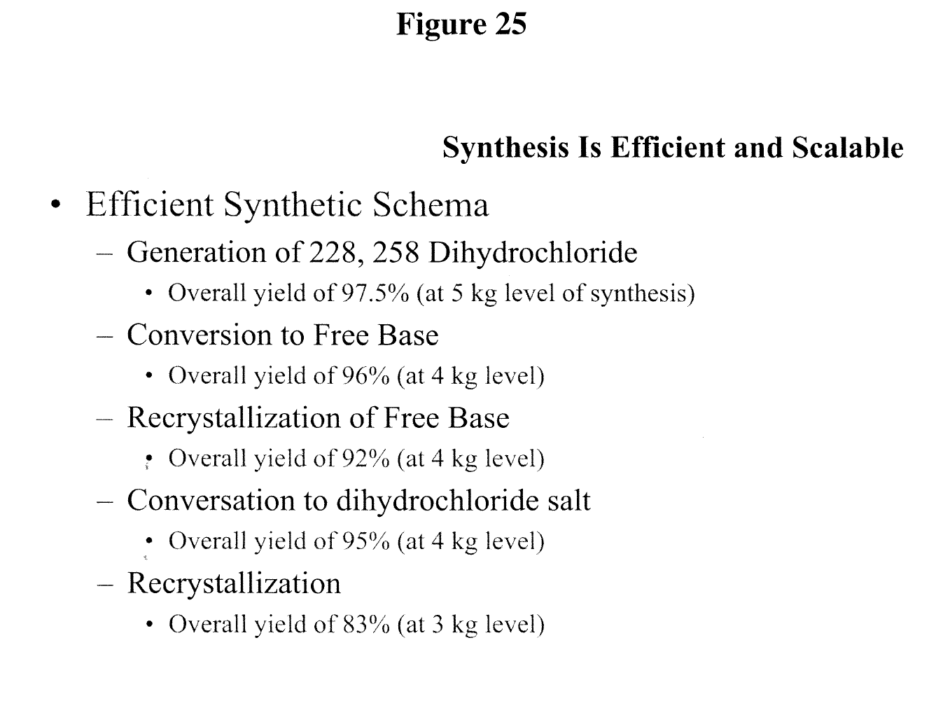

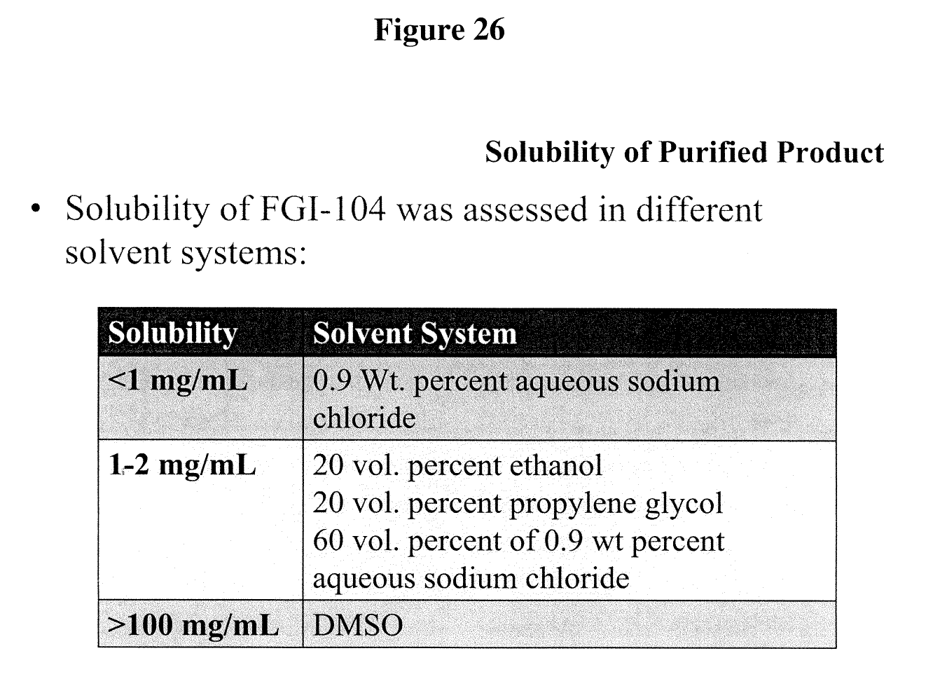

For agricultural or pharmaceutical distribution, a synthesis scheme conformable to Good Laboratory Practice is required. Such a GLP compliant scheme is set forth in FIG. 24, which yields the active agent from commonly available starting materials in three short steps. The yields of this synthesis route are appreciable, as set forth in FIG. 25. The resulting purified product, suitable for pharmaceutical preparation for humans or animals, is soluble in a variety of solvent systems, as reflected in FIG. 26. When reference is made herein to FGI-104 compounds, reference is intended to the compounds themselves, pharmaceutically acceptable salts such as hydrochloride salts, amide preparations, and pharmaceutically acceptable solutions and other formulations, such as suspensions.

The invention of this application has been disclosed in the context of multiple examples, as well as generic discussion and formulae. Except where specifically indicated by the terms of the claims set forth below, the Examples are not intended to be limiting. Those of skill in the art, given the examples and the assay information set forth herein, with the knowledge of the type of anti-viral performance possible, would easily arrive at the identification of other suitable compounds and methods of administration without the exercise of inventive faculty to arrive at treatments for viral infection.

What is claimed is:

1. A compound of the formula 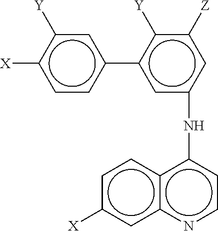
wherein each substituent X is independently H or an electron donating group, which may be selected from the group including chloro chloro or other halogen, hydroxy, alkoxy (—OR), aryloxy (—OAr), trialkylammonium (—NR₃+), alkylamido (—NHCOR, —NRCOR′), arylamido (—NHCOAr, —NRCOAr, —NArCOAr), arylcarbamoyl (—NHCOOAr, —NRCOOAr), alkylcarbamoyl (—NHCOOR, —NRCOOR′), cyano (—CN), nitro (—NO₂), ester (—COOR, —COOAr), or alkyl halo, each substituent Y is independently H, alkyl of 1-4 carbon atoms, hydroxy, alkoxy oe methylene and wherein
substituent Z is a di-or-tri akly amino, or alkyl di or tri amino, optionally substituted with a halogen moiety,
further wherein said compound, when administered in effective amounts to a mammalian cell infected with a virus, inhibits viral infection and replication in said cell without cytotoxic effects for said cell.

2. The compound of claim 1, wherein said compound is selected from the group consisting of R15, R16, R17, R18, R19, R20, R21, R22, R23, R24, R25, R26, R27, R28, R29, R30, R31 and R32.

3. The compound of claim 2, wherein said compound is R19 or R24.

4. The compound of claim 3, wherein said compound is R24.

5. A method of treating viral infection in a mammalian cell, comprising administering an effective amount of a compound of claim 1 to said cell, therapeutically or prophylactically, wherein said virus is Ebola virus, Marburg virus, human immunodeficiency virus (HIV), Hepatitis B virus (HBV), Hepatitis C virus (HCV), Dengue fever virus, porcine reproductive and respiratory syndrome (PRRS) virus, bovine corona virus, influenza virus, an alphavirus, cowpox virus, West Nile virus, respiratory syncytial virus (RSV), parainfluenza virus (PIV), Human metapneumovirus (HMPV) Punta Toro virus, a circovirus, EIAV, bluetongue, and foot and mouth disease (FMD) viruses.

6. The method of claim 5, wherein said virus is Ebola, Marburg, Influenza, HBV, HCV, Dengue fever, West Nile or RSV.

7. The method of claim 5, wherein said virus is PRRS, porcine corona virus, bovine corona virus, an alphavirus, cowpox virus, Punta Toro virus, porcine circovirus, bovine circovirus, EIAV, bluetongue virus, or FMD virus.

8. A method of treating viral infection in a mammalian cell, comprising administering an effective amount of a compound claim 1 therapeutically or prophylactically, wherein said virus is a virus of Group IV, Group V, Group VI, or Group VII.

9. The method of claim 8, wherein said compound is R19 or R24.

10. The method of claim 5, wherein said cell is part of a cell culture, and said compound is administered in vitro.

11. The method of claim 5, wherein said cell is part of a mammalian host's body, and said compound is administered to said host in vivo.

12. The method of claim 8 wherein said cell is part of a mammalian host's body, and said compound is administered to said host in vivo.

13. A method of treating a viral infection in a mammalian host, comprising interfering with interaction between a virus causing said infection and TSG101 protein of said host, wherein said method of interfering comprises administering to said host a compound of claim 1, and wherein viral replication and budding of said virus is effected in said host in part by interaction with TSG101.

14. A pharmaceutical composition, comprising an amount of the compound of claim 1 in a pharmaceutically acceptable carrier, wherein said compound of claim 1 is present in amounts effective to treat a mammalian host with a viral infection such that said infection is attenuated when administered to said mammalian host.

15. The composition of claim 14, wherein said compound is R19 or R24.

16. A method of treating viral infection in a mammal in need of same, which comprises administering a compound of claim 1 to said mammal in such fashion and amount as to interfere with post replication maturation and release of viral replicons of said virus.
